(19) United States
(12) Patent Application Publication
(10) Pub. No.: US 2004/0198789 A1
(43) Pub. Date: Oct. 7, 2004

(54) Lercanidipine/ARB/diuretic therapeutic combinations

(75) Inventors: Amedeo Leonardi, Milan (IT); Abraham Sartani, Arese (IT); Giorgio Sironi, Pieve Emanuele (IT)

Correspondence Address: DARBY & DARBY P.C., P. O. BOX 5257, NEW YORK, NY, 10150-5257, US

(73) Assignee: Recordati Ireland Limited

(21) Appl. No.: 10/791,148

(22) Filed: Mar. 1, 2004

Related U.S. Application Data
(60) Provisional application No. 60/450,782, filed on Feb. 28, 2003.
(60) Provisional application No. 60/450,864, filed on Feb. 28, 2003.
(60) Provisional application No. 60/478,285, filed on Jun. 13, 2003.

(51) Int. Cl. A61K 31/41

(52) U.S. Cl. 514/381

(57) ABSTRACT

Lercanidipine is used in the preparation of a medicament for the treatment of hypertension in combination with the prior, concurrent or post-administration of an angiotensin II receptor blocker (ARB), and optionally, a diuretic. Compositions containing both lercanidipine and the ARB, and optionally, a diuretic are contemplated. Single, once-a-day dosage forms are preferred. A method for the treatment of hypertension in a sub-population of patients with tachycardia is also recited.

This application claims priority under 35 U.S.C. § 119(e) of U.S. provisional applications serial No. 60/450,864, filed Feb. 28, 2003; 60/450,782, filed Feb. 28, 2003; and 60/478,285, file Jun. 13, 2003, each of which is incorporated by reference herein in its entirety.

### FIELD OF THE INVENTION

The present invention contemplates a method for treating hypertension with a combination of lercanidipine and an angiotensin II receptor blocker (ARB), and, optionally, a diuretic. The present invention also contemplates a method for treating hypertension in a sub-population of patients with tachycardia with a combination of lercanidipine and an ARB, and optionally, a diuretic.

### BACKGROUND OF THE INVENTION

Hypertension is one of the most common cardiovascular disease states. In the United States, over 50 million people have been diagnosed with hypertension (which is defined as a blood pressure greater than or equal to 140/90 mm Hg). Elevated arterial pressure can cause pathological changes in the vasculature and hypertrophy of the left ventricle. Due to the damage that can be produced by hypertension, it is proposed to be the principal cause of stroke, myocardial infarction, and sudden cardiac death. Additionally, it is believed to be a major contributor to cardiac failure, renal insufficiency, and dissecting aneurysm of the aorta.

The renin-angiotensin-aldosterone system is an important regulator of arterial pressure. The inactive angiotensinogen peptide is converted to the pro-peptide angiotensin I by the enzyme renin. Angiotensin I then is converted to the active angiotensin II form by the angiotensin converting enzyme. Angiotensin II then acts through a variety of receptor mediated mechanisms, such as increasing the total peripheral resistance and inhibiting the excretion of sodium and water by the kidneys, to increase arterial pressure.

Current Treatments for Hypertension

Angiotensin II receptor blockers (ARBs) are a class of active agents used in the treatment of hypertension. ARBs block the vasoconstrictor and aldosterone-secreting effects of angiotensin II by selectively blocking the binding of angiotensin II to the AT₁ receptor in many tissues, such as smooth muscle and the adrenal gland. ARBs include irbesartan (Avapro®), valsartan (Diovan®), candesartan cilexetil (“candesartan”/Atacand®), telmisartan (Micardis®), olmesartan medoxomil (“olmesartan”/Benicar®), losartan potassium (“losartan”/Cozaar®), and eprosartan mesylate (“eprosartan”/Teveten®). (Physicians' Desk Reference, 57th edition, 2003).

The recommended daily dosages of ARBs when used as monotherapy for the treatment of hypertension is as follows: irbesartan—150 to 300 mg; valsartan—80 to 320 mg; candesartan—8 to 32 mg; telmisartan—40 to 80 mg; olmesartan—20 to 40 mg; losartan—25 to 100 mg, and eprosartan 600-800 mg.

ARBs are commercially available. Irbesartan is available from Bristol-Myers Squibb and Sanofi-Synthelabo under the trade name Avapro® in dosages of 75, 150 and 300 mg. Valsartan is available from Novartis under the trade name Diovan® in dosages of 40, 80, 160 and 320 mg. Telmisartan is available from Boehringer Ingelheim under the trade name Micardis® in dosages of 20, 40 and 80 mg. Olmesartan is available from Sankyo under the trade name Benicar® in dosages of 5, 20 and 40 mg. Losartan is available from Merck under the trade name Cozaar® in dosages of 25, 50 and 100 mg. Candesartan cilexetil is available from AstraZeneca under the trade name Atacand® in dosages of 4 mg, 8 mg, 16 mg and 32 mg. Eprosartan is available from Bioavail under the trade name Teveten® in dosages of 400 and 600 mg.

Another class of active agents that is used for the treatment of hypertension is calcium antagonists. These active agents influence the influx of calcium ions into cells, especially smooth muscle cells. Inhibition of calcium influx produces a relaxation of smooth muscles, such as those around the arteries and veins, which leads to a decrease in the observed hypertension. Such active agents as well as their hypotensive activity are described in a number of publications and patent applications.

In addition, diuretics are often added as adjunct therapy to ARBs for the treatment of hypertension. In fact, candesartan and telmisartan are co-formulated with the diuretic hydrochlorothiazide (HCT) for the treatment of hypertension. Diuretics reduce the amount of fluid in the blood stream by lowering the amount of salt and water in your body, which helps to reduce blood pressure. There are three main types of diuretic drugs, thiazide diuretics, potassium-sparing diuretics, and loop diuretics. These are discussed further below. However, while it is known that co-administration of an ARB and a diuretic is effective therapy for the prevention and treatment of hypertension (see WO 03/087045, which claims a combination of valsartan, amlodipine and HCT), and WO 2003/09704 discloses triple a combination of an ARB, a calcium channel blocker, and a diuretic, neither of these published applications disclose lercanidipine as the calcium channel blocker, nor do they disclose specific combinations with superior results.

Several pharmacological rationales can be advanced for combining an ARB with a calcium antagonist to treat hypertension. For example, the fact that multiple physiologic systems participate in blood pressure control has been proposed as a major reason why individual active agents decrease in efficacy over time. The pharmacological intervention on one of these systems is believed to trigger counterregulatory mechanisms. A combination of treatments increases the number of mechanisms potentially capable of reducing an elevated blood pressure and reduces the rate and magnitude of the adverse events produced by each drug. Further, the addition of one agent may counteract some deleterious effects of the other. Therefore a low-dose combination of two different agents reduces the risk of dose-related adverse reaction while still allowing sufficient blood pressure reduction.

In addition to pharmacological advantages, combination therapy has been requested to meet evolving guidelines that look for more aggressive treatment of blood pressure. For example, recent World Health Guidelines recommend a diastolic blood pressure lower than 85 mm Hg and a systolic blood pressure lower than 130 mm Hg in younger patients and in diabetic patients.

Fixed combinations offer the possibility of administering a combination of active agents in a single dosage form. Such a form will likely increase patient compliance. That is, such a dosage form will likely increase a patient's adherence to a therapeutic scheme and will increase the success of such a treatment.

Additionally, a number of patients are nonresponsive to one or more of the available monotherapies, and some patients are not responsive to known combination therapies. There is no way at present to predict whether these patients will be responsive to therapy using a new combination of active ingredients. It has been calculated that, overall, 30-50% of patients are non-responders to monotherapy (this average does not include data of patients taking lercanidipine). The combined therapy of lercanidipine with candesartan has been reported (Aranda, et al., J. Hypertension. June 2000; 18 (Suppl. 2):S 152).

There is a continuing need for safe and effective combination anti-hypertensive treatments that have a long lasting, selective mechanism of action with few side effects.

Tachycardia

Hypertension and cardiac arrhythmias commonly coexist in many patients, and both need to be managed appropriately. Furthermore, hypertensive left ventricular hypertrophy could cause a wide variety of ventricular arrhythmias, which could end in sudden cardiac arrest (Greenberg et al., J Clin Hypertens (Greenwich) 2000; 2(1):14-19; Hennersdorf et al., J Hypertens. 2001; 19(2): 167-77). Heart rate is electrically regulated by the sin θ-atrial (SA) node of the right atrium to be about 60-100 beats per minute. When the ventricular chambers beat too quickly, the arrhythmia is known as ventricular tachycardia (VT). When VT occurs, the ventricles may not be able to fill with enough blood to supply the body with sufficient amounts of oxygen rich blood. Symptoms of VT include feeling faint, passing out, dizziness, or a pounding in the chest. The most common electrical therapy for VT is implantation of a device known as an Implantable Cardioverter Defibrillator or ICD. The ICD applies an electric shock to the heart muscle to interrupt or disrupt the fast rhythm. The electric shock may be in the form of specially timed pacemaker pulses (unfelt by the patient) or by high voltage shock.

In addition, a side effect of some dihydropyridine calcium channel blockers can be reflex tachycardia. Heart palpitations were reported in about 0-9% of patients who were administered lercanidipine (the range is a result of statistically created amplitude to reflect the margin of error). The palpitations manifested either at the beginning of treatment, or during dose escalation, disappearing upon cessation of medication. However, of the calcium channel blockers, lercanidipine showed one of the lowest, if not the lowest, incidence of tachycardia as a side effect. Sustained-release administration, such as in a depot formulation, was also shown to abrogate this side effect (Sada et al., Nippon Yakurigaku Zasshi. 2003; 122(6): 539-47; Harada et al., Circ. J. 2003; 67(2): 139-45; and van Zweiten, Blood Press. Suppl. 1998; 2:5-9).

Accordingly, there is a need in the art to mitigate the tachycardia associated with hypertension, or associated with dihydropyridine calcium channel blockers used to treat hypertension, since tachycardia or other arrhythmia can result in cardiac events and death.

### SUMMARY OF THE INVENTION

The present invention contemplates methods for treating hypertension in multiple classes of patients. A first class of patients are those that are responders to monotherapy with an ARB or lercanidipine, but who suffer from dose-related side effects and for whom it would be desirable to decrease the dosage amount of the active agent used in monotherapy. In other words, these active agents produce antihypertensive activity and decrease the patient's blood pressure by the predetermined increment. A combination of an ARB and lercanidipine is particularly suitable for such patients.

Accordingly, in one aspect, the present invention is directed to a method for treating hypertension in a patient in need thereof, the method comprising administering to the patient a first amount of an ARB and a second amount of lercanidipine, where the amounts in combination are effective to reduce blood pressure in the patient by at least a predetermined increment and preferably to restore blood pressure to within acceptable limits; where at least one of the first amount and the second amount is either ineffective to produce a reduction in blood pressure in the patient, or the reduction in blood pressure is less than the predetermined increment. In other words, the amounts of the two agents employed in the combination would each be suboptimal or sub-threshold (i.e., producing a decrease in blood pressure less than the predetermined amount or totally ineffective if administered as monotherapy). In a preferred embodiment both the first amount and the second amount are individually ineffective to produce a reduction in blood pressure in the patient, or only partially effective (the reduction in blood pressure is less then the predetermined increment).

A second patient class are of patients who are nonresponders to monotherapy. In these patients, the active agent or agents alone do not produce antihypertensive activity or stop doing so after a period of treatment. In another aspect, the present invention encompasses a method for treating hypertension in a nonresponder patient in need thereof, the method comprising administering to the patient a first amount of an ARB and a second amount of lercanidipine where the amounts in combination are effective to reduce blood pressure in the patient by at least a predetermined increment, and preferably to restore blood pressure to within acceptable limits. The patient would usually have been previously determined not to respond or to respond insufficiently or to stop responding to monotherapy with lercanidipine or an ARB or even with another single antihypertensive agent. This embodiment is particularly desirable for those patients that are resistant to lercanidipine monotherapy. Lercanidipine generally works quite well, so patients resistant to lercanidipine monotherapy can be difficult to treat.

A third class of patients are of patients who are partial responders to monotherapy and combination therapy. Monotherapy or combination therapy produces an antihypertensive effect in these patients, but the therapy does not decrease the blood pressure by the predetermined increment. Higher doses of the individual agents do not produce the desired effect of decreasing blood pressure by the predetermined amount, and may produce undesirable side effects. In another aspect, the present invention encompasses a method for treating hypertension in a partial responder patient in need thereof, the method comprising administering to the patient a first amount of ARB and a second amount of lercanidipine wherein the amounts in combination are effective to reduce blood pressure in the patient by at least a predetermined increment, and preferably to restore blood pressure to within acceptable limits, wherein each of the first amount and the second amount if administered alone is ineffective to produce a reduction in blood pressure by the predetermined increment.

A fourth class of patients includes those that are responders to monotherapy but have been previously determined (or are expected) to become nonresponders or partial responders over time. Conventionally, patients in this class, upon becoming nonresponders, would then require a monotherapy involving higher dosage amounts of the same active agent or would need a change of medication to another active agent to treat hypertension (i.e., reduce blood pressure by the predetermined increment). However, it should be noted that these patients may not further respond to increased dosages due to maximal efficacy of the compound having been reached. The cause for such a change in a patient's response also may be a compensatory (counterregulatory) mechanism or another cause.

In yet another aspect, the present invention encompasses a method for treating hypertension in a patient within the fourth class, where the patient has been previously determined to be responsive to monotherapy with lercanidipine or an ARB the method comprising administering to the patient a composition comprising a first combination therapy amount of lercanidipine and a second combination therapy amount of an ARB, where the combination therapy amounts are in combination effective to reduce the patient's blood pressure by at least the predetermined increment, and preferably restore blood pressure to within acceptable limits. In a preferred embodiment, the amounts of lercanidipine and ARB are sub-threshold amounts of each agent that would not be effective in monotherapy. The lower doses would reduce the side effects that may present with the therapeutic doses prescribed for monotherapy.

It is a further object of the invention to administer a diuretic in combination with the above-described treatment methods. The diuretics can be thiazide diuretics, potassium sparing diuretics, and loop diuretics. In a preferred embodiment, the diuretic co-administered with lercanidipine and the ARB is a thiazide diuretic.

Yet another class of patients that can advantageously be treated with the method of the present invention are patients experiencing or diagnosed with hypertension having tachycardia. Lastly, in principle, the present invention can be employed with naive patients although the regulatory authority guidelines do not encourage such a practice.

Compositions and dosage forms are further contemplated by the present invention.

The compositions and methods described herein have the potential advantages of allowing treatment with suboptimal amounts of one or both agents, sub-threshold amounts of at least one active agent, allowing greater tolerability in patients sensitive to the active agent, of allowing for synergism, i.e., superadditivity between active agents, of allowing for sustained long term efficacy of treatment and for sustained dosaging throughout a dosage period or for achieving regulation of blood pressure that was elevated, i.e., severe hypertension.

Moreover, the compositions and methods described herein have potential of being of increased effectiveness in treatment or decreased side effects, compared to other combinations of lercanidipine and other classes of active agents or combinations of lercanidipine with other ARBs.

In other embodiments, the invention provides any of the aforementioned methods or compositions with the proviso that the ARB is not candesartan.

### BRIEF DESCRIPTION OF THE DRAWINGS

FIG. 1 shows the effects on systolic blood pressure (SBP) observed in a rat model of essential hypertension following administration of vehicle, lercanidipine, irbesartan, or the combination of lercanidipine and irbesartan.

FIG. 2 shows the effects on diastolic blood pressure (DBP) observed in a rat model of essential hypertension following administration of vehicle, lercanidipine, irbesartan, or the combination of lercanidipine and irbesartan.

FIG. 3 shows the change in systolic blood pressure (SBP) observed in a rat model of essential hypertension following administration of vehicle, lercanidipine, irbesartan, or the combination of lercanidipine and irbesartan. Significance values “p” are for the observed differences between control treatment with vehicle and a particular treatment.

FIG. 4 shows the change in diastolic blood pressure (DBP) observed in a rat model of essential hypertension following administration of vehicle, lercanidipine, irbesartan, or the combination of lercanidipine and irbesartan. Significance values “p” are for the observed differences between control treatment with vehicle and a particular treatment.

FIG. 5 shows the effects on heart rate (HR) observed in a rat model of essential hypertension following administration of vehicle, lercanidipine, irbesartan, or the combination of lercanidipine and irbesartan.

FIG. 6 shows the change in heart rate (HR) observed in a rat model of essential hypertension following administration of vehicle, lercanidipine, irbesartan, or the combination of lercanidipine and irbesartan. Significance values “p” are for the observed differences between control treatment with vehicle and a particular treatment.

FIG. 7 shows the effects on systolic blood pressure (SBP) observed in a rat model of essential hypertension following administration of vehicle, lercanidipine, olmesartan, or the combination of lercanidipine and olmesartan.

FIG. 8 shows the effects on diastolic blood pressure (DBP) observed in a rat model of essential hypertension following administration of vehicle, lercanidipine, olmesartan, or the combination of lercanidipine and olmesartan.

FIG. 9 shows the change in systolic blood pressure (SBP) observed in a rat model of essential hypertension following administration of vehicle, lercanidipine, olmesartan, or the combination of lercanidipine and olmesartan. Significance values “p” are for the observed differences between control treatment with vehicle and a particular treatment.

FIG. 10 shows the change in diastolic blood pressure (DBP) observed in a rat model of essential hypertension following administration of vehicle, lercanidipine, olmesartan, or the combination of lercanidipine and olmesartan. Significance values “p” are for the observed differences between control treatment with vehicle and a particular treatment.

FIG. 11 shows the effects on heart rate (HR) observed in a rat model of essential hypertension following administration of vehicle, lercanidipine, olmesartan, or the combination of lercanidipine and olmesartan.

FIG. 12 shows the change in heart rate (HR) observed in a rat model of essential hypertension following administration of vehicle, lercanidipine, olmesartan, or the combination of lercanidipine and olmesartan. Significance values “p” are for the observed differences between control treatment with vehicle and a particular treatment.

FIG. 13 shows the effects on systolic blood pressure (SBP) observed in a rat model of essential hypertension following administration of vehicle, lercanidipine, valsartan, or the combination of lercanidipine and valsartan.

FIG. 14 shows the effects on diastolic blood pressure (DBP) observed in a rat model of essential hypertension following administration of vehicle, lercanidipine, valsartan, or the combination of lercanidipine and valsartan.

FIG. 15 shows the change in systolic blood pressure (SBP) observed in a rat model of essential hypertension following administration of vehicle, lercanidipine, valsartan, or the combination of lercanidipine and valsartan. Significance values “p” are for the observed differences between control treatment with vehicle and a particular treatment.

FIG. 16 shows the change in diastolic blood pressure (DBP) observed in a rat model of essential hypertension following administration of vehicle, lercanidipine, valsartan, or the combination of lercanidipine and valsartan. Significance values “p” are for the observed differences between control treatment with vehicle and a particular treatment.

FIG. 17 shows the effects on heart rate (HR) observed in a rat model of essential hypertension following administration of vehicle, lercanidipine, valsartan, or the combination of lercanidipine and valsartan.

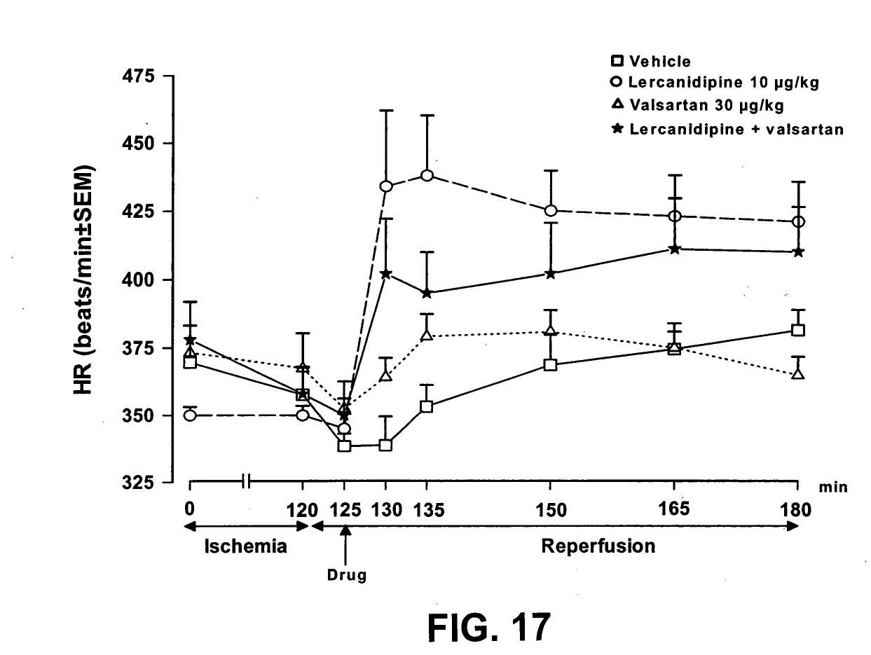

FIG. 18 shows the change in heart rate (HR) observed in a rat model of essential hypertension following administration of vehicle, lercanidipine, valsartan, or the combination of lercanidipine and valsartan. Significance values “p” are for the observed differences between control treatment with vehicle and a particular treatment.

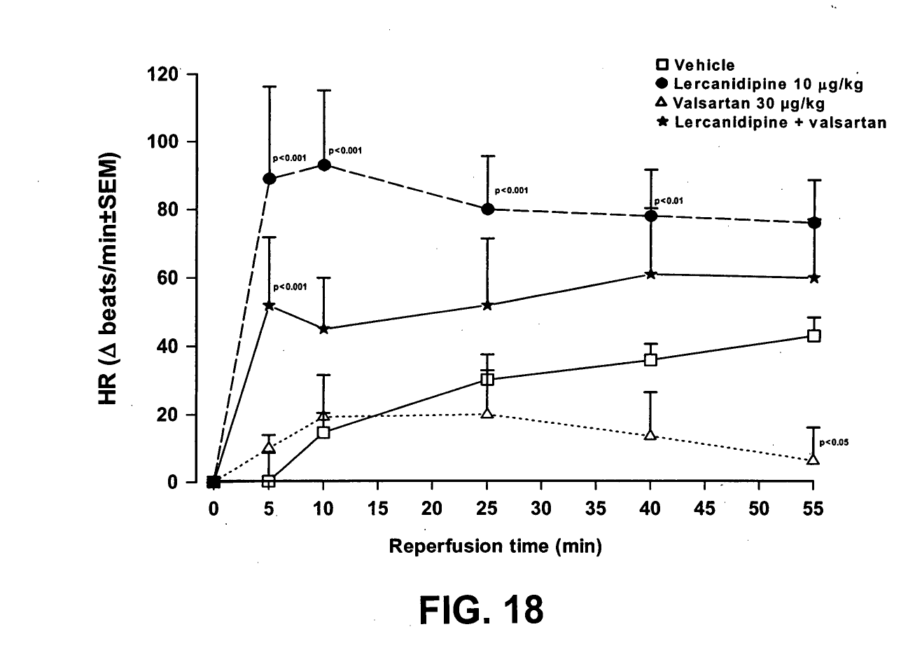

### DETAILED DESCRIPTION

The present invention describes methods for treating hypertension in multiple classes of patients. A first class of patients are those that are responders to monotherapy with ARBs or calcium channel blockers (CCB), i.e., lercanidipine, but who suffer from side-effects and for whom it would be desirable to decrease the dosage amount of the active agent used in monotherapy. In other words, these active agents produce antihypertensive activity and decrease the patient's blood pressure by the predetermined increment. A combination of ARB and lercanidipine is particularly suitable for such patients. Moreover, although these patients may be partial responders, they are not necessarily regulated after combined therapy, necessitating higher doses of one or both active agents.

Another class of patients is a hypertensive population that is afflicted with tachycardia upon treatment with an antihypertensive as monotherapy. It has unexpectedly been discovered that the combination of lercanidipine with an ARB mitigates drug-induced tachycardia.

Angiotensin Type II Receptor Antagonists (ARBS)

Olmesartan medoxomil (“olmesartan”), 2,3-dihydroxy-2-butenyl 4-(1-hydroxy-1-methylethyl)-2-propyl-1-[-(o-1H-tetrazol-5-ylphenyl)benzyl]imidazole-5-carboxylate cyclic 2,3-carbonate, is an angiotensin II receptor (AT, subtype) antagonist described in U.S. Pat. No. 5,616,599. It is a prodrug that is hydrolyzed to the corresponding acid, 4-(1-hydroxy-1-methylethyl)-2-propyl-1-[p-(o-1H-tetrazol-5-ylphenyl)benzyl]imidazole-5-carboxylic acid (olmesartan free acid), during absorption from the gastrointestinal tract. Following oral administration of olmesartan, peak serum concentrations of olmesartan free acid occur within about 1-2 hours. There is virtually no metabolism of olmesartan free acid, approximately 35 to 50% is cleared in the urine and the remainder is eliminated in feces. Olmesartan free acid is a specific competitive antagonist of the AT, receptor with a much greater affinity (more than 12,500-fold) for AT₁ receptor compared to AT₂ receptor and no antagonist activity. Blockade of the AT₁ receptor removes the negative feedback of angiotensin II on renin secretion. The resulting increase in plasma renin activity and circulating angiotensin II, however, does not overcome the positive effects of olmesartan for treating hypertension. Olmesartan free acid does not inhibit ACE or renin or affect other hormone receptors or ion channels known to be involved in regulation of blood pressure.

The recommended starting dosage of olmesartan as monotherapy for essential hypertension is 20 mg once per day, with drug titration to 40 mg daily. Several weeks of therapy may be required to achieve optimal blood pressure reduction for a patient. A lower initial dose is recommended in patients with depletion of intravascular volume or salt. No dosage adjustment is necessary in elderly patients or in patients with hepatic impairment or mild to severe renal impairment. Safety and effectiveness have not been established in children. Treatment with olmesartan is well tolerated with an incidence of adverse events similar to placebo.

Irbesartan (2-butyl-3-[[2′-(1H-tetrazol-5-yl)[1,1′-biphenyl]-4-yl]methyl]-1,3-diazaspiro[4.4]non-1-en-4-one) is an angiotensin II receptor (AT, subtype) antagonist described in U.S. Pat. No. 5,270,317. Following oral administration, peak serum concentrations of irbesartan occur within about 1.5-2 hours. Irbesartan is metabolized via glucuronide conjugation and oxidation. Irbesartan is a specific competitive antagonist of the AT₁ receptor with a much greater affinity (more than 8500-fold) for AT₁ receptor compared to AT₂ receptor and no agonist activity. Blockade of the AT₁ receptor removes the negative feedback of angiotensin II on renin secretion. The resulting increase in plasma renin activity and circulating agiotensin II, however, does not overcome the positive effects of irbesartan for treating hypertension. Irbesartan does not inhibit ACE or renin or affect other hormone receptors or ion channels known to be involved in regulation of blood pressure.

The recommended starting dosage of irbesartan as monotherapy for essential hypertension is 150 mg once per, with drug titration 300 mg per day. Several weeks of therapy may be required to achieve optimal blood pressure reduction for a patient. A lower initial dose of 75 mg is recommended in patients with depletion of intravascular volume or salt. No dosage adjustment is necessary in elderly patients or in patients with hepatic impairment or mild to severe renal impairment. Safety and effectiveness have not been established in children under 6 years old. Children 6-12 years old may reasonably be started on a dosage of 75 mg once daily, with titration to 150 mg daily. Treatment with irbesartan is well tolerated with an incidence of adverse events similar to placebo.

Valsartan (N-(1-oxopentyl)-N-[[2′-(1H-tetrazol-5-yl)[1,1′-biphenyl]-4-yl]methyl]-L-valine) is a nonpeptide, orally active, and specific angiotensin II antagonist acting on the AT₁ receptor subtype, described in U.S. Pat. No. 5,399,578. Following oral administration, peak plasma concentration is reached 2 to 4 hours after dosing. When administered as an oral solution it is primarily recovered in feces (about 83% of dose) and urine (about 13% of dose). The recovery is mainly as unchanged drug, with only about 20% of dose recovered as metabolites. The primary metabolite, accounting for about 9% of dose, is valery 4-hydroxy valsartan. The enzyme(s) responsible for valsartan metabolism have not been identified but do not seem to be CYP 450 isozymes. Valsartan has much greater affinity (about 20,000 fold) for the AT₁ receptor than for the AT₂ receptor. The increased plasma levels of angiotensin II following AT₁ receptor blockade with valsartan may stimulate the unblocked AT₂ receptor. Because valsartan does not inhibit ACE (kininase II), it does not affect the response to bradykinin. Valsartan does not bind to or block other hormone receptors or ion channels known to be important in cardiovascular regulation.

The recommended starting dose of valsartan is 80 mg or 160 mg once daily when used as monotherapy in patients who are not volume-depleted. The antihypertensive effect is substantially present within 2 weeks and maximal reduction is generally attained after 4 weeks. No initial dosage adjustment is required for elderly patients, for patients with mild or moderate renal impairment, or for patients with mild or moderate liver insufficiency. Safety and effectiveness in pediatric patients have not been established. The overall incidence of adverse experiences reported with valsartan was similar to placebo.

Telmisartan (4′-[1,4′-dimethyl-2[-propyl[2,6′-bi-1H-benzimidazol]-1′-yl)methyl]-[1,1′-biphenyl]-2-carboxylic acid) is a nonpeptide angiotensin II AT₁ antagonist described in U.S. Pat. No. 5,591,762. Following oral administration, peak serum concentrations are reached in 0.5-1 hour. Telmisartan is metabolized by a P450 cytochrome enzyme-independent mechanism by conjugation to form inactive acylgucouronide. Following either intravenous or oral administration, most of the administered dose (>97%) was eliminated unchanged in feces via biliary excretion. Telmisartan has much greater affinity (>3000 fold) for the AT₁ receptor than for the AT₂ receptor. Telmisartan does not inhibit ACE, therefore, it does not affect the response to bradykinin (which is inhibited by ACE inhibitors). Telmisartan does not bind to or block other hormone receptors or ion channels known to be important in cardiovascular regulation.

The recommended starting dose of telmisartan as monotherapy for hypertension is 40 mg, once a day, with doses up to 80 mg/day. Additional therapy with a diuretic is recommended if doses above 80 mg/day are warranted. Most of the anti-hypertensive effect is apparent after two weeks, and maximal reduction is generally attained after four weeks. No initial dosage adjustment is required for elderly patients or for patients with mild or moderate renal impairment. Patients with liver insufficiency should be closely supervised. Safety and effectiveness in pediatric patients have not been established. The overall incidence of adverse experiences reported with telmisartan have been mild and transient in nature, and have only infrequently required discontinuation of therapy.

Losartan (2-butyl-4-chloro-1-[p-(o-1H-tetrazol-5-ylphenyl)-benzyl]imidazole-5-methanol monopotassium salt) is also a nonpeptide molecule and is described in U.S. Pat. No. 5,138,069. Losartan is metabolized by cytochrome P450 enzymes, and is converted, in part, to an active carboxylic acid metabolite that is responsible for most of the ARB activity. Following administration, mean peak concentrations of losartan and its metabolite are achieved in 1 hour and in 3-4 hours, respectively. About 14% of orally administered losartan is converted to the active metabolite. The terminal half-life of losartan is about 2 hours and of the metabolite is about 6-9 hours. Both losartan and its principal active metabolite do not exhibit any partial agonist activity at the AT₁ receptor and have much greater affinity (about 1000-fold) for the AT₁ receptor than for the AT₂ receptor. Losartan does not inhibit ACE, nor does it bind to or block other hormone receptors or ion channels known to be important in cardiovascular regulation.

The recommended initial dose of losartan is 50 mg, once a day, with 25 mg/day recommended if the patient is concurrently taking diuretics. The maximum required dose is about 100 mg/day. Individualized dosing is recommended. The antihypertensive effect of losartan is usually seen after one week, but can be delayed up to 3-6 weeks. No initial adjustment is necessary for elderly patients, or patients with renal impairment. A lower starting dose is recommended for patients with hepatic insufficiency. Losartan has not been investigated in patients under the age of 18. The overall incidence of adverse effects observed with losartan is similar to placebo.

Candesartan ((±)-1-[[(cyclohexyloxy)carbonyl]oxy]ethyl 2-ethoxy-1]]2′-(1H-tetrazol-5-yl) [1,1′[-biphenyl]4-yl]methyl]1H-benzimidazole-7-carboxylate) is a prodrug described in U.S. Pat. No. 5,196,444 for the treatment of hypertension. Candesartan is rapidly and completely bioactivated by ester hydrolysis during absorption by the gastrointestinal tract to candesartan free acid, a selective, achiral AT₁ subtype antagonist. Candesartan free acid has a much greater affinity (<10,000) for the AT₁ receptor than the AT₂ receptor. Candesartan undergoes mild hepatic metabolism by O-deethylation to an inactive metabolite. Following oral administration, peak serum concentration is achieved after 3-4 hours. Candesartan is not preferred for combination with lercanidipine in the absence of a diuretic for essential hypertension.

Individual dosing of candesartan is recommended, with a recommended starting dose of 16 mg/day as monotherapy. Administration can be from about 8 mg to 32 mg once or twice daily. Most of the antihypertensive effect is seen after about 2 weeks, and maximal blood pressure reduction is seen at about 4-6 weeks. No differences were seen when candesartan was administered to patients with hepatic insufficiency, and no dosage adjustment is required for patients with renal insufficiency or elderly patients. The effects of candesartan have not been evaluated in a pediatric population.

Eprosartan ((E)-2-butyl-1 (p-carboxybenzyl)-α-2-thienylmethylimidazole-5-acrylic acid-Teveten®) is described in U.S. Pat. No. 5,185,351. Eprosartan mesylate is a reversible inhibitor of the AT₁ receptor. Eprosartan affinity for the AT₁ receptor is about 1000 times more than for the AT₂ receptor. Eprosartan is eliminated by biliary and renal excretion, primarily as an unchanged compound. There are no known active metabolites. Plasma concentrations peak at about 1-2 hours after oral administration.

The usual recommended starting dose of eprosartan is 600 mg once a day when used as monotherapy, but daily doses can range from 400-800 mg. No initial dosing adjustment is needed for the elderly or those with renal impairment if the maximum dose does not exceed 600 mg per day. Eprosartan is well-tolerated for most people up to 1200 mg per day.

Lercanidipine

Another class of active agents that is used for the treatment of hypertension is calcium antagonists. These active agents influence the influx of calcium ions into cells, especially smooth muscle cells. Inhibition of calcium influx produces a relaxation of smooth muscles, such as those around the arteries and veins, which leads to a decrease in the observed hypertension. Such active agents as well as their hypotensive activity are described in a number of publications and patent applications.

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist with long duration of action and high vascular selectivity. Its mechanism of antihypertensive activity is due to a direct relaxant effect on vascular smooth muscle, thus lowering total peripheral resistance. The recommended starting dose of lercanidipine HCl (“lercanidipine”) immediate release tablets as monotherapy is 10 mg daily by oral route, with a drug titration to 20 mg daily (for immediate release). Daily administration of 80 mg per day is permitted for modified-release formulations of lercanidipine, which, due to the modified release profile, prevents excessive plasma levels of lercanidipine at any given time point. Lercanidipine is rapidly absorbed following oral administration with peak plasma levels occurring 2-3 hours following dosing. Elimination is essentially via the hepatic route. In comparison to other calcium antagonists, lercanidipine is characterized by gradual onset and long-lasting duration of action despite decreasing plasma levels. In vitro studies show that isolated rat aorta response to high K⁺ may be attenuated by lercanidipine, even after the preparate has been repeatedly washed out during 6 hours. Lercanidipine is commercially available from Recordati S.p.A. (Milan, Italy) and has been described along with methods for making it and resolving it into individual enantiomers in U.S. Pat. Nos. 4,705,797; 5,767,136; 4,968,832; and 5,696,139.

Clinical studies have shown that lercanidipine 10 mg daily (typically titrated to 20 mg daily in patients not responding or responding inadequately to the 10 mg dose) provides a sustained pharmacological action and a significant antihypertensive effect. In hypertensive patients the onset of lercanidipine action is gradual and the drug has a consistent and sustained blood pressure lowering effect throughout the dosage interval. The gradual and smooth antihypertensive effect has been recently confirmed also by using the “Smoothness Index”, as described in Omboni and Zanchetti, Hypertension, 1998, 16:1831-8. The analysis of a large population of hypertensive patients has documented that lercanidipine is a very well tolerated drug, for the most part, with few and/or moderate side effects, including tachycardia (rare), palpitations and lower extremities edema. However, only about 0-9% of patients have reported tachycardia as a side effect. In man, lercanidipine is contraindicated (as are all dihydropyridines) in patients with unstable angina or recent (<1 month) myocardial infarction.

Diuretics

Thiazide diuretics directly inhibit sodium and calcium reabsorbtion and augment calcium absorption in the early distal convoluted tubule of the kidney. Excess sodium, calcium, and water reduce extracellular volume in mild to moderate congestive heart failure. Thiazide diuretics potentiate anti-hypertensive agents by about ⅓ to 1/2 in an effort to reduce blood pressure and overall fluid volume. The increased serum concentrations of calcium allow for the relaxation of arterial smooth muscle that, in turn, reduces peripheral vascular resistance. Thiazide diuretics possess anti-hypertensive properties because of the direct vasodilatation of arterioles, altered sodium balance, and the reduction in fluid volume. Thiazide diuretics are not the drug of choice if massive amount of diuresis are necessary or if the patient has a history of intolerance to sulfa containing medications.

Commonly used thiazide diuretics in the United States are Aquatensen® (methyclothiazide), Diucardin® (hydroflumethiazide), Diulo® (metolazone), Diuril® (chlorothiazide), Enduron® (methyclothiazide), Esidrix® (hydrochlorothiazide), Hydro-chlor® (hydrochlorothiazide; HCT), Hydro-D® (hydrochlorothiazide; HCT), HydroDIURIL® (hydrochlorothiazide), Hydromox® (quinethazone), Hygroton® (chlorthalidone), Metahydrin® (trichlormethiazide), Microzide® (hydrochlorothiazide), Mykrox® (metolazone), Naqua® (trichlormethiazide), Naturetin® (bendroflumethiazide), Oretic® (hydrochlorothiazide), Renese® (polythiazide), Saluron® (hydroflumethiazide), Thalitone® (chlorthalidone), Trichlorex® (trichlormethiazide), and Zaroxolyn® (metolazone). An additional thiazide or thiazide-like diuretic includes Lozol® (indapamide), benzylhydrochlorothiazide, cyclopenthiazide, polythiazide, ethiazide, and cyclothiazide.

Preferred diuretics are, without limitation, hydrochlorothiazide (HCT), 6-chloro-3,4-dihydro-2H-1,2,4,-benzothiadiazin-7-sulfonamide 1,1-dioxide and chorthalidone.

Potassium-sparing diuretics are mild diuretics that act upon the distal convoluting tubule to inhibit sodium exchange for potassium. Gradually, sodium and water are excreted in the urine, and potassium is conserved. Aldactone (spironolactone) is a synthetic steroid that is similar to aldosterone and acts as an antagonist by competing for aldosterone binding sites. Inhibition of aldosterone leads to the excretion of sodium and the retention of potassium in the distal portion of the kidney nephrons. Other members of the potassium-sparing diuretic group do not alter aldosterone binding, but work primarily by impairing the exchange of potassium and sodium in the distal convoluting tubule. These agents act as a slight anti-hypertensive and potentiate anti-hypertensive medications. Potassium-sparing diuretics can be given in conjunction with potassium-pitching diuretics in an attempt to prevent hypokalemia and the complications involved with that particular electrolyte imbalance.

Commonly used potassium-sparing diuretics used in the United States include Aldactone® (spironolactone), Dyrenium® (triamterene), and Midamor® (amiloride).

Loop diuretics relieve excess extracellular fluid volume and to regulate vascular osmolarity. Loop diuretics are the most potent and expedient diuretics available, and they inhibit the reabsorbtion of sodium, chloride, and potassium ions in the ascending loop of Henle in the kidney. Loop diuretics also cause renal vasodilatation and a transient rise in glomerular filtration rate. The combination of increased renal blood flow and the prevention of the sodium-potassium-chloride co-transport system permits secretion of large volumes of fluid and electrolytes. Loop diuretics have systemic hemodynamic effects: increased venous capacitance (which reduces left ventricular filling pressures or preload), increased ejection fraction (an indicator of improved ventricular function), decreased systemic and peripheral vascular resistance (reduced pulmonary, organ, and extracellular edema or afterload) which all allow for a reduction in blood pressure and cardiac workload.

Commonly used brand names in the United States are bumetanide (Bumex®, Budema®, Bumedyl®, Burinex®, Busix®, Butinat®, Cambiex®, Farmadiuril®, Fontego®, Fordiuran®, Lunetoron®, Miccil®, Pendock®, Poliurene®, Segurex®); torsemide_(Demadex®); ethacrynic acid (Edecrin®, Edecril®, Hydromedin®, Reomax®); furosemide (Lasix®, Myrosemide®, Aldic®, Aluzin®, Aquamide®, Aquasin®, Arasemide®, Bioretic®, Cetasix®, Detue®, Dirine®, Discoid®, Disemide®, Diural®, Diuresal®, Diurolasa®, Diusil®, Dranex®, Dryptal®, Durafurid®, Edenol®, Errolon®, Eutensin®, Fluidrol®, Franyl®, Frumex®, Frusedan®, Frusema®, Frusemid®, Frusetic®, Frusid®, Frusix®, Furantril®, Furetic®, Furex®, Furmide®, Furocot®, Furodiurol®, Furomide M.D®, Furorese®, Furoside®, Furosix®, Furovite®, Fusid®, Golan®, Hissuflux®, Kofuzo®, Kutrix®, Lasemid®, Lasiletten®, Lasilix®, Laxur®, Liside®, Lo-Aqua®, Luramide®, Marsemid®e, Nadis®, Nelsix®, Nildema®, Novosemide®, Odemase®, Oedemex®, Promedes®, Radisemide®, Radonna®, Rasitol®, Retep®, Ro-Semide®, Rose-40®, Salinex®, Salurid®, Seguril®, Sigasalur®, Trofurit®, Uremide®, Urenil®, Uresix®, Urian®, Uritol®, Yidoli, Furosan®, and Furoter®).

Since they are not very effective alone, the potassium-sparing diuretics are often typically combined into a single tablet or capsule with another diuretic—usually HCT. In addition, diuretics are often put together into a single tablet or capsule with drugs from other classes of antihypertensives. For example, HCT has been combined with ACE inhibitors, beta blockers, and ARBs. Candesartan cilexetil and telmisartan are both marketed in a combined formulation with HCT for the treatment of hypertension. Candesartan cilexetil comes in either 16 mg or 32 mg candesartan cilexetil, with 12.5 mg HCT, while telmisartan comes in 40 mg or 80 mg telmisartan with 12.5 mg HCT.

### Definitions

As used herein, the term “hypertension” refers to abnormally high arterial blood pressure, when compared to prior blood pressure readings, and the abnormally high value is maintained over a specified time period. Conventionally, the time period is 3-6 months. The increase may be observed in systolic pressure, diastolic pressure, or both. Conventionally, hypertension is defined as a blood pressure of equal to or greater than 140/90 mm Hg. Blood pressure may be measured by any method known in the art. Such methods include, but are not limited to direct arterial puncture, oscillometry, Doppler ultrasonography, and a sphygmomanometer. In a preferred embodiment, blood pressure is measured with a sphygmomanometer. While the person taking the measurement listens to the pulse of the patient and watches the sphygmomanometer gauge, two measurements (systolic pressure and diastolic pressure) are recorded. Blood pressure is measured in millimeters of mercury (mm Hg).

The terms “systolic” and “systolic pressure” refer to the pressure induced by the contraction of the heart by which the blood is forced onward and the circulation kept up. The terms “diastolic” and “diastolic pressure” refer to the pressure induced by the dilatation of the cavities of the heart during the period in which they fill with blood. Typically, blood pressure is expressed as two numbers separated by a slash, where the first number is the systolic pressure and the second number is the diastolic pressure. As mentioned above, the pressure is conventionally expressed as mm Hg.

Blood pressure in normal and hypertensive adults is typically categorized as follows:

| | Systolic | Diastolic |
|---|---|---|
| Category | Pressure, mmHg | Pressure, mmHg |
| Normal | <120 | <80 |
| Prehypertension | 120-139 | 80-89 |
| Stage 1 Hypertension | 140-159 | 90-99 |
| Stage 2 Hypertension | >160 | >100 |

Source: The Seventh Report of the Joint National Committee on Prevention, Detection, Evaluation, and treatment of High Blood Pressure (JAMA,289 (19) 2560-72 (2003))

The term “antihypertensive activity” refers to the effect of an active agent to lower the blood pressure of a patient with hypertension. In one embodiment, the blood pressure is decreased by at least 20 mm Hg for systolic pressure or by at least 10 mm Hg for diastolic pressure. In another embodiment, the antihypertensive activity refers to the effect of an active agent to lower the blood pressure by at least 20 mm Hg for systolic pressure and by at least 10 mm Hg for diastolic pressure. The active agent may or may not decrease the blood pressure in a person that does not have hypertension or may not decrease blood pressure in all persons with hypertension or may not achieve blood pressure regulation in all patients who experience a decrease in blood pressure. In a preferred embodiment, the active agent decreases a patient's blood pressure to below 140/90 mm Hg (i.e., regulates the blood pressure).

The term “active agent” or “active ingredient” refers to a compound that produces a pharmacological effect that leads to a physiological change. As used herein, the active agents are antihypertensive agents, such as lercanidipine and olmesartan which are employed in the combination treatment of the invention. Conventionally, an active agent is considered as having an antihypertensive effect if it decreases either systolic or diastolic blood pressure by at least 10 mm Hg.

The term “predetermined increment” refers to the minimum reduction in blood pressure that is needed for a patient to decrease blood pressure to or below a predetermined limit, preferably 140/90. Thus, an active agent which at a dosage tolerated by the patient achieves reduction by a predetermined increment is considered effective to treat hypertension in the specific patient, and the patient is considered responsive to this agent (also known as a “responder,” as defined below). In other words, if an active agent decreases blood pressure by a predetermined increment in one patient (i.e., has sufficient antihypertensive activity in the patient) but does not decrease blood pressure by the predetermined increment in another patient (i.e., does not have sufficient antihypertensive activity in the patient), then the first patient is responsive to the treatment (a “responder,” as defined below) but the second patient is not (a “nonresponder,” as defined below). The decrease in blood pressure can be in the systolic pressure, diastolic pressure, or both. Reduction in diastolic blood pressure is a particularly desirable result.

As used herein, the term “responder” refers to a patient that has previously responded to a treatment for hypertension involving administration of a particular active agent (or combination of active agents) in a particular amount or amounts. In other words, the active agent or active agents have “antihypertensive activity” and reduce the patient's blood pressure by the “predetermined increment”. A determination of responsiveness to an antihypertensive regimen may require administration of a particular agent in a particular amount and frequency for a period of time, usually 1 month for ARBs and calcium antagonists. Such treatments include, but are not limited to, administration of ARBs, calcium channel blockers, beta blockers, ACE inhibitors and diuretics. The phrase “responsive to monotherapy” refers to patients who are administered only one active agent (monotherapy) and the monotherapy achieves a reduction in blood pressure by the “predetermined increment” as that term is defined above. In a specific embodiment, the antihypertensive activity is defined as at least a decrease of 20 mm Hg in systolic pressure or as at least a decrease of 10 mm Hg for diastolic pressure.

The term “nonresponder” refers to a patient who has been determined not to have responded to treatment for hypertension with a particular agent or combination of agents, i.e., for whom the regimen has not achieved a reduction in blood pressure. In other words, the active agent or active agents do not have antihypertensive activity in the patient, and therefore the patient's blood pressure is not decreased by the predetermined increment. The term encompasses patients who do not undergo any decrease in blood pressure upon treatment with, e.g., lercanidipine alone or ARB alone or diuretic alone.

The term “partial responder” refers to a patient for whom a particular active agent (or combination of active agents), in a particular amount or amounts, produces “antihypertensive activity” in the patient but does not decrease blood pressure by the “predetermined increment”. Increases in the amount of active agent (or combination of active agents) may or may not further decrease the blood pressure of these patients. The term encompasses patients that respond only insufficiently, i.e., exhibit some decrease in blood pressure, but short of the “predetermined increment” (to below 140/90 mm Hg). Generally, in those patients the amount of antihypertensive agent needs to be increased. But this may bring on or aggravate side effects.

The terms “suboptimal” or “sub-threshold” amounts of active agent for monotherapy refer to amounts of active agent that are insufficient to decrease blood pressure by the predetermined increment. A “suboptimal” amount is an amount that is effective to decrease the blood pressure but does not decrease it sufficiently to achieve regulation in a patient who would be a responder if administered an effective amount. A “sub-threshold” amount is an amount that provides no effect on individuals who would respond to an effective dose. “Suboptimal” or “sub-threshold” amounts may well vary from patient to patient. A patient who fails to achieve a decrease in blood pressure by the predetermined increment (or at all) upon administration of a given dosage of active agent has either been administered a “suboptimal” or “sub-threshold” amount of active agent or may, alternatively, be a non-responder to the active agent. “Suboptimal” or “sub-threshold” amounts of active agent may be distinguished from the case of administration to a non-responder by increasing the administered dosage of active agent. In the case where a patient fails to achieve a decrease in blood pressure by the predetermined increment due to administration of a “suboptimal” or “sub-threshold” amount of active agent, administration of an increased dosage of active agent will cause the patient to achieve a decrease in blood pressure by the predetermined increment. In the case where a patient fails to achieve a decrease in blood pressure by the predetermined increment due to said patient being a non-responder, increasing the dosage of active agent will not cause the patient to achieve a decrease in blood pressure by the predetermined increment.

As used herein, the term “monotherapy” refers to the administration of a single active agent to treat hypertension.

The term “efficacy of treatment” refers to the potency of a drug in treating hypertension.

The term “in combination” refers to the concomitant administration of two (or more) active agents for the treatment of a single disease state. As used herein, the active agents may be combined and administered in a single dosage form, may be administered as separate dosage forms at the same time, or may be administered as separate dosage forms that are administered alternately or sequentially on the same or separate days. In one preferred embodiment of the present invention, the active agents are combined and administered in a single dosage form. In other preferred embodiments, the active agents are administered in separate dosage forms (e.g., wherein it is desirable to vary the amount of one but not the other). The single dosage form may include additional active agents for the treatment of the disease state. In a preferred embodiments, the single dosage form comprises lercanidipine and an ARB. Further preferred are embodiments wherein the single dosage form comprises lercanidipine and an ARB, with the proviso that the ARB is not candesartan. In other preferred embodiments, a single dosage from comprises lercanidipine, an ARB, and a diuretic.

The term “combination therapy” refers to administration of at least two active ingredients “in combination” for the treatment of hypertension. In the present invention lercanidipine and ARB may be further combined with one or more additional active ingredients, e.g., a diuretic and/or a β-receptor blocker and/or an ACE inhibitor, without limitation.

Diuretics include but are not limited to all those listed above. ACE inhibitors include the following examples, which are non-limiting: captopril, enalapril, lisinopril, quinapril, fosinopril, ramipril, cilazapril, spirapril, benazepril and perindopril.

β-receptor blockers include the following examples, which are non-limiting: acebutolol HCl, atenolol, betaxolol HCl, carvedilol, esmolol HCl, labetalol HCl, levobunolol HCl, metoprolol, nadolol, pindonol, propranolol HCl, sotalol HCl, timolol and timolol maleate.

“Tachycardia” refers to an increased heart rate, i.e., above the normal range of about 60-100 beats per minute. Abnormal heart rates can range from about 100-400 beats per minute and can be life threatening. Tachycardia can arise from atrial fibrillation (AF), where the heartbeat is irregular and rapid due to the upper chambers, or atria, beating about four times faster than normal. AF can lead to other rhythm problems, chronic fatigue and congestive heart failure. By contrast, ventricular tachycardia (VT) refers to sudden rapid heartbeats originating in the ventricles, and are the most dangerous of arrhythmias. VT can lead to ventricular fibrillation (VF), which is characterized by irregular and chaotic rapid heartbeats. Because the fibrillating ventricular muscle cannot contract and pump blood to the brain and vital organs, VF is the number one cause of sudden cardiac death. Without immediate emergency treatment of an electric shock to restore normal rhythm, an individual loses consciousness within seconds and dies within minutes.

### Combination Therapy

### Formulations and Compositions

The active agents of the combinations of the present invention may be formulated into a single pharmaceutical composition or each can be administered in different pharmaceutical compositions.

Pharmaceutical compositions may include optional additives, such as a pharmaceutically acceptable carrier or diluent, a flavor, a sweetener, a preservative, a dye, a binder, a suspending agent, a dispersing agent, an alkalizing agent, buffering agents, a wetting agent, coating agents, a glidant, an anticaking agent, a colorant, a disintegrant, an excipient, a diluent, a lubricant, a plasticizer, an edible oil or any combination of two or more of the foregoing.

Suitable pharmaceutically acceptable carriers or diluents include, but are not limited to, ethanol; water; glycerol; aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, povidone, starch; gelatin; natural sugars, such as glucose, sucrose and lactose; corn sweeteners; natural and synthetic gums, such as acacia, tragacanth, xantan gum, guar gum, vegetable gum, and sodium alginate; carboxymethylcellulose; polyethylene glycol; waxes; and the like.

Suitable disintegrants include, but are not limited to, starch such as corn starch, methyl cellulose, agar, bentonite, sodium starch glycolate, sodium crosscarmellose, crosspovidone and the like.

Suitable lubricants include, but are not limited to, sodium oleate, sodium stearate, magnesium stearate, sodium benzoate, stearic acid, gliceryl behenate, and the like.

A suitable suspending agent is, but is not limited to, bentonite.

Suitable dispersing and suspending agents include, but are not limited to, synthetic and natural gums, such as vegetable gum, tragacanth, xantan gum, guar gum, acacia, alginate, dextran, sodium carboxymethylcellulose, methylcellulose, polyvinyl-pyrrolidone and gelatin.

Suitable edible oils include, but are not limited to, cottonseed oil, sesame oil, coconut oil and peanut oil.

Suitable alkalizing agents, include, but are not limited to, trolamine, meglumine, sodium carbonate, sodium bicarbonate, sodium hydroxide, diethanolamine.

Suitable buffering agents, include, but are not limited to, sodium phosphate mono and dibasic, potassium phosphate, sodium citrate, citric acid, sodium lactate, lactic acid.

Suitable wetting agents, include, but are not limited to, Polysorbate 20, Polysorbate 40, Polysorbate 60, Polysorbate 80, Poloxamer, Sodium lauryl sulphate, sorbaitan monooleate, sorbitan monopalmitate, sorbitan monostearate, Tyloxapol, Polyoxyl 35 castor oil, Polyoxyl 40 hydrogenated castor oil, Polyoxyl 40 Stearate. Suitable coating agents include, but are not limited to, Hypromellose, Methylcellulose, Polyvinil acetate phthalate, Cellulose acetate, cellulose acetate phtalate, Ethylcellulose, Hydroxypropyl cellulose, Hypromellose phthalate, Shellac, Methacrylic acid copolymers.

Suitable glidants include, but are not limited to, Calcium silicate, Magnesium silicate, Silicon colloidal dioxide, talc.

Suitable anticaking agent include, but are not limited to, Calcium silicate, Magnesium silicate, Silicon colloidal dioxide.

Examples of additional additives include, but are not limited to, sorbitol; talc; stearic acid; and dicalcium phosphate. Commercially available preparations containing the ARBs and lercanidipine can be used. Naturally, if the preparation contains more than one active ingredient, the amounts in the combination may have to be adjusted downwards.

Unit dosage forms. The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage, therefore the preferred dosage forms are tablets, pills, and capsules. However, parenteral preparations also are preferred.

Solid unit dosage forms may be prepared by mixing the active agents of the present invention with a pharmaceutically acceptable carrier and any other desired additives as described above. The mixture is typically mixed until a homogeneous mixture of the active agents of the present invention and the carrier and any other desired additives is formed, i.e., until the active agents are dispersed evenly throughout the composition. In this case, the compositions can be formed as dry or moist granules.

Tablets or pills can be coated or otherwise compounded to form a unit dosage form which has delayed and/or prolonged action, such as time release and sustained release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents, include, but are not limited to, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For liquid dosage forms, the active substances or their physiologically acceptable salts are brought into solution, suspension or emulsion (comprising micro- and nano-emulsions), optionally with the usually employed substances such as solubilizers, emulsifiers or other auxiliaries. Solvents for the active combinations and the corresponding physiologically acceptable salts, can include water, physiological salt solutions or alcohols, e.g. ethanol, propane-diol or glycerol. Additionally, sugar solutions such as fructose, sucrose, glucose or mannitol solutions may be used. A mixture of the various solvents mentioned may further be used in the present invention.

A transdermal dosage form also is contemplated by the present invention. Transdermal forms may be a diffusion-driven transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery system. Transdermal dosage forms may be used for timed release and sustained release of the active agents of the present invention.

Pharmaceutical compositions and unit dosage forms of the present invention for administration parenterally, and in particular by injection, typically include a pharmaceutically acceptable carrier, as described above. Liquid compositions may be, for example and without limitation, solutions, microemulsions, or fat emulsions. A preferred liquid carrier is vegetable oil. Injection may be, for example, intravenous, epidural, intrathecal, intramuscular, intraluminal, intratracheal, or subcutaneous.

The active agents also can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers as targetable drug carriers. Such polymers include, but are not limited to, polyvinyl-pyrrolidone, pyran copolymer, polyhydroxypropylmethacryl-amidephenol, polyhydroxy-ethylaspartamidephenol, and polyethyl-eneoxideopolylysine substituted with palmitoyl residues.

Lercanidipine can be formulated as a physiologically acceptable salt, e.g., a salt with an inorganic or organic acid such as e.g. HCl, HBr, H₂SO₄, benzenesulfonic maleic acid, fumaric acid, tartaric acid and citric acid.

Active agents are preferably administered as single dosage forms comprising lercanidipine and ARB or lercanidipine, ARB and diuretic. Single dosage forms may, without limitation, comprise lercanidipine, ARB and optionally diuretic together in admixture. Single dosage forms may alternatively, without limitation, comprise a capsule containing two or more sets of multiparticulates having different active agents, one set containing lercanidipine, another set an ARB, and optionally, third set containing diuretic. The multiparticulates themselves can be made by any of the conventional methods, including extrusion spheronisation, high shear granulation, non-pareil seeds, etc. Dosage forms of this type are suitable for oral use.

### COMPOSITION EXAMPLES

TABLE A
Lercanidipine/ARB Formulations

| Ingredient (mg/tablet) | 1 | 2 | 3 | 4 | 5 |
|---|---|---|---|---|---|
| Lercanidipine HCl | 10 | 10 | 10 | 10 | 10 |
| Olmesartan | 20 | — | — | — | — |
| Irbesartan | — | 150 | — | — | — |
| Valsartan | — | — | 160 | — | — |
| Telmisartan | — | — | — | 40 | — |
| Losartan | — | — | — | — | 50 |
| Lactose monohydrate | 102 | 102 | 102 | 102 | 102 |
| Microcrystalline cellulose | 40 | 80 | 82 | 46 | 49 |
| Sodium bicarbonate | 8 | 16 | 16 | 9 | 10 |
| Sodium starch glycolate | 20 | 40 | 41 | 23 | 25 |
| Povidone K30 | 8 | 16 | 16 | 9 | 10 |
| Magnesium stearate | 2 | 4 | 4 | 2 | 2 |

TABLE B
Lercanidipine/ARB/Hydrochlorothiazide Formulations

| Ingredient (mg/tablet) | 6 | 7 | 8 | 9 | 10 | 11 |
|---|---|---|---|---|---|---|
| Lercanidipine HCl | 10 | 10 | 10 | 10 | 10 | 10 |
| Hydrochlorothiazide | 20 | 20 | 20 | 20 | 20 | 20 |
| Olmesartan | 20 | — | — | — | — | — |
| Irbesartan | — | 150 | — | — | — | — |
| Valsartan | — | — | 160 | — | — | — |
| Telmisartan | — | — | — | 40 | — | — |
| Losartan | — | — | — | — | 50 | — |
| Candesartan | — | — | — | — | — | 20 |
| Lactose monohydrate | 82 | 82 | 82 | 82 | 82 | 82 |
| Microcrystalline | 40 | 80 | 82 | 46 | 49 | 40 |
| cellulose | | | | | | |
| Sodium bicarbonate | 8 | 16 | 16 | 9 | 10 | 8 |
| Sodium starch glycolate | 20 | 40 | 41 | 23 | 25 | 20 |
| Povidone K30 | 8 | 16 | 16 | 9 | 10 | 8 |
| Magnesium stearate | 2 | 4 | 4 | 2 | 2 | 2 |

TABLE C
Lercanidipine/ARB/Chlorthalidone Formulations

| Ingredient (mg/tablet) | 12 | 13 | 14 | 15 | 16 | 17 |
|---|---|---|---|---|---|---|
| Lercanidipine HCl | 10 | 10 | 10 | 10 | 10 | 10 |
| Chlorthalidone | 25 | 25 | 25 | 25 | 25 | 25 |
| Olmesartan | 20 | — | — | — | — | — |
| Irbesartan | — | 150 | — | — | — | — |
| Valsartan | — | — | 160 | — | — | — |
| Telmisartan | — | — | — | 40 | — | — |
| Losartan | — | — | — | — | 50 | — |
| Candesartan | — | — | — | — | — | 20 |
| Lactose monohydrate | 77 | 77 | 77 | 77 | 77 | 77 |
| Microcrystalline cellulose | 40 | 80 | 82 | 46 | 49 | 40 |
| Sodium bicarbonate | 8 | 16 | 16 | 9 | 10 | 8 |
| Sodium starch glycolate | 20 | 40 | 41 | 23 | 25 | 20 |
| Povidone K30 | 8 | 16 | 16 | 9 | 10 | 8 |
| Magnesium stearate | 2 | 4 | 4 | 2 | 2 | 2 |

A film coated tablet may be prepared using the cores described above in Tables A-C and using the composition described in Table D.

TABLE D
Coating for tablet formulations shown in Tables A-C

| Ingredient | Amount* |
|---|---|
| Hypromellose | 1.91 mg |
| Talc | 0.15 mg |
| Titanium dioxide | 0.60 mg |
| Macrogol 6000 | 0.30 mg |
| Ferric oxide | 0.04 mg |

*per 100 mg of core

### Dosing and Administration

The pharmaceutical composition or unit dosage forms of the present invention may be administered by a variety of routes such as intravenous, intratracheal, subcutaneous, oral, parenteral, buccal, sublingual, ophthalmic, pulmonary, transmucosal, transdermal, and intramuscular. Unit dosage forms also can be administered in intranasal form via topical use of suitable intranasal vehicles, or via transdermal routes, using those forms of transdermal skin patches known to those of ordinary skill in the art. Oral administration is preferred.

The pharmaceutical composition or unit dosage forms of the present invention may be administered to an animal, preferably a human being, in need of antihypertensive treatment. The pharmaceutical composition or unit dosage form of the present invention may be administered according to a dosage and administration regimen defined by routine testing in light of the guidelines given above in order to obtain optimal antihypertensive activity (especially for patients who are partial responders or nonresponders to conventional monotherapy or to other combination therapies) and a decreased in blood pressure by the predetermined increment while minimizing toxicity or side-effects for a particular patient. However, such fine turning of the therapeutic regimen is routine in light of the guidelines given herein.

The dosage of the active agents of the present invention may vary according to a variety of factors such as underlying disease state, the individual's condition, weight, sex and age and the mode of administration. For oral administration, the pharmaceutical compositions can be provided in the form of scored or unscored solid unit dosage forms. For the ARBs, the dosage forms comprise 5 mg, 20 mg, or 40 mg for olmesartan; 75 mg, 150 mg, or 300 mg for irbesartan; 80 mg, 120 mg or 160 mg for valsartan; 20 mg, 40 mg or 80 mg for telmisartan; 12.5 mg, 25 mg or 50 mg for losartan; and 8 mg, 16 mg or 32 mg for candesartan, for the symptomatic adjustment of the dosage to the patient to be treated. Preferably, the ARB dosage forms comprise 20 or 40 mg for olmesartan; 150 or 300 mg for irbesartan; 40 or 80 mg for telmisartan; 25 or 50 mg for losartan; and 16 or 32 mg for candesartan. For lercanidipine, the dosage forms comprise 2.5, 5.0, 10.0, 20.0, 40.0, 60.0 or 80.0 mg for the symptomatic adjustment of the dosage to the patient to be treated. Preferably, the lercanidipine dosage forms comprise 2.5, 5.0, 10.0, 20.0 or 40.0 mg. The 80 mg dosage is preferably administered in the form of a modified release tablet. The modified release decreases initial peak concentration and provides sustained blood levels for a prolonged period of time, e.g., 24 h.

For ARB/lercanidipine combination therapy according to the invention, the active agents may initially be provided as separate dosage forms until an optimum dosage combination and administration regimen is achieved. Therefore, the patient may be titrated to the appropriate dosages for his/her particular hypertensive condition, including the presence or absence of tachycardia at the same time as hypertension or after treatment with lercanidipine. After the appropriate dosage of each of the active agents is determined to achieve a decrease of the blood pressure by the predetermined increment without untoward side effects, the patient then may be switched to a single dosage form containing the appropriate dosages of each of the active agents, or may continue with a dual dosage form.

In the present invention, the amount of ARB administered to a patient in the combination therapy with lercanidipine (for the treatment of both hypertension and tachycardia), except for irbesartan and candesartan, will be preferably within the range of 10 to 100 mg per day in a single or two divided doses. For irbesartan, the preferred dosage administered in combination will be from about 75-300 mg per day, and for candesartan, the preferred dosage in combination will be from about 8-32 mg per day. More preferably, the amount of ARB, except for irbesartan and candesartan, will be 10-40 mg per day. For irbesartan, the more preferred amount administered will be 75-150 mg per day, and for candesartan, the more preferred dose will be 8-16 mg per day. The amount of lercanidipine will be preferably within the range of 2.5-80 mg, more preferably, 10-40 mg, and most preferably 10-20 mg.

The amount of lercanidipine will be preferably within the range of 2.5-80 mg, more preferably, 10-40 mg, and most preferably, 10-20 mg. As stated above, 80 mg daily is tolerated when administered in a modified release formulation. The preferred combinations for olmesartan are: (i) 10 mg of olmesartan and 2.5 mg of lercanidipine, (ii) 10 mg of olmesartan and 5 mg of lercanidipine, (iii) 10 mg of olmesartan and 10 mg of lercanidipine, (iv) 10 mg of olmesartan and 20 mg of lercanidipine (v) 10 mg of olmesartan and 40 mg of lercanidipine (vi) 10 mg of olmesartan and 60 mg of lercanidipine (vii) 10 mg of olmesartan and 80 mg of lercanidipine (viii) 20 mg of olmesartan and 2.5 mg of lercanidipine, (ix) 20 mg of olmesartan and 5 mg of lercanidipine, (x) 20 mg of olmesartan and 10 mg of lercanidipine, (xi) 20 mg of olmesartan and 20 mg of lercanidipine, (xii) 20 mg of olmesartan and 40 mg of lercanidipine, (xiii) 20 mg of olmesartan and 60 mg of lercanidipine, (xiv) 20 mg of olmesartan and 80 mg of lercanidipine, (xv) 40 mg of olmesartan and 2.5 mg of lercanidipine, (xvi) 40 mg of olmesartan and 5 mg of lercanidipine, (xvii) 40 mg of olmesartan and 10 mg of lercanidipine, (xviii) 40 mg olmesartan and 20 mg lercanidipine, (xix) 40 mg of olmesartan and 40 mg of lercanidipine, (xx) 40 mg of olmesartan and 60 mg of lercanidipine, or (xxi) 40 mg of olmesartan and 80 mg of lercanidipine.

The preferred combinations for irbesartan are: (i) 75 mg of irbesartan and 2.5 mg of lercanidipine, (ii) 75 mg of irbesartan and 5 mg of lercanidipine, (iii) 75 mg of irbesartan and 10 mg of lercanidipine, (iv) 75 mg of irbesartan and 20 mg of lercanidipine, (v) 75 mg of irbesartan and 40 mg of lercanidipine, (vi) 75 mg of irbesartan and 60 mg of lercanidpine, (vii) 75 mg of irbesartan and 80 mg of lercanidipine (viii), 150 mg of irbesartan and 2.5 mg of lercanidipine, (ix) 150 mg of irbesartan and 5 mg of lercanidipine, (x) 150 mg of irbesartan and 10 mg of lercanidipine, (xi) 150 mg of irbesartan and 20 mg of lercanidipine, (xii) 150 mg of irbesartan and 40 mg of lercanidipine, (xiii) 150 mg of irbesartan and 60 mg of lercanidipine, (xiv) 160 mg of irbesartan and 80 mg of lercanidipine, (xv) 300 mg of irbesartan and 2.5 mg of lercanidipine, (xvi) 300 mg of irbesartan and 5 mg of lercanidipine, (xvii) 300 mg of irbesartan and 10 mg of lercanidipine, (xviii) 300 mg irbesartan and 20 mg lercanidipine, (xix) 300 mg of irbesartan and 40 mg of lercanidipine, (xx) 300 mg of irbesartan and 60 mg of lercanidipine, or (xxi) 300 mg of irbesartan and 80 mg of lercanidipine.

The preferred combinations of valsartan are: (i) 80 mg of valsartan and 2.5 mg of lercanidipine, (ii) 80 mg of valsartan and 5 mg of lercanidipine, (iii) 80 mg of valsartan and 10 mg of lercanidipine, (iv) 80 mg of valsartan and 20 mg of lercanidipine, (v) 80 mg of valsartan and 40 mg of lercanidipine, (vi) 80 mg of valsartan and 60 mg of lercanidipine, (vii) 80 mg of valsartan and 80 mg of lercanidipine, (viii) 120 mg of valsartan and 2.5 mg of lercanidipine, (ix) 120 mg of valsartan and 5 mg of lercanidipine, (x) 120 mg of valsartan and 10 mg of lercanidipine, (xi) 120 mg of valsartan and 20 mg of lercanidipine, (xii) 120 mg of valsartan and 40 mg of lercanidipine, (xiii) 120 mg of valsartan and 60 mg of lercanidipine, (xiv) 120 mg of valsartan and 80 mg of lercanidipine, (xv) 160 mg of valsartan and 2.5 mg of lercanidipine, (xvi) 160 mg of valsartan and 5 mg of lercanidipine, (xvii) 160 mg of valsartan and 10 mg of lercanidipine, (xviii) 160 mg valsartan and 20 mg lercanidipine, (xix) 160 mg of valsartan and 40 mg of lercanidipine, (xx) 160 mg of valsartan and 60 mg of lercanidipine, or (xxi) 160 mg of valsartan and 80 mg of lercanidipine.

The preferred combinations of losartan are: (i) 12.5 mg of losartan and 2.5 mg of lercanidipine, (ii) 12.5 mg of losartan and 5 mg of lercanidipine, (iii) 12.5 mg of losartan and 10 mg of lercanidipine, (iv) 12.5 mg of losartan and 20 mg of lercanidipine, (v) 12.5 mg of losartan and 40 mg of lercanidipine, (vi) 12.5 mg of losartan and 60 mg of lercanidipine, (vii) 12.5 mg of losartan and 80 mg of lercanidipine, (viii) 25 mg of losartan and 2.5 mg of lercanidipine, (ix) 25 mg of losartan and 5 mg of lercanidipine, (x) 25 mg of losartan and 10 mg of lercanidipine, (xi) 25 mg of losartan and 20 mg of lercanidipine, (xii) 2e5 mg of losartan and 40 mg of lercanidipine, (xiii) 25 mg of losartan and 60 mg of lercanidipine, (xiv) 25 mg of losartan and 80 mg of lercanidipine, (xv) 50 mg of losartan and 2.5 mg of lercanidipine, (xvi) 50 mg of losartan and 5 mg of lercanidipine, (xvii) 50 mg of losartan and 10 mg of lercanidipine, (xviii) 50 mg losartan and 20 mg lercanidipine, (xix) 50 mg of losartan and 40 mg of lercanidipine, (xx) 50 mg of losartan and 60 mg of lercanidipine, or (xxi) 50 mg of losartan and 80 mg of lercanidipine.

The preferred combinations of candesartan are: (i) 8 mg of candesartan and 2.5 mg of lercanidipine, (ii) 8 mg of candesartan and 5 mg of lercanidipine, (iii) 8 mg of candesartan and 10 mg of lercanidipine, (iv) 8 of candesartan and 20 mg of lercanidipine, (v) 8 mg of candesartan and 40 mg of lercanidipine, (vi) 8 mg of candesartan and 60 mg of lercanidipine, (vii) 8 mg of candesartan and 80 mg of lercanidipine, (viii) 16 mg of candesartan and 2.5 mg of lercanidipine, (ix) 16 mg of candesartan and 5 mg of lercanidipine, (x) 16 mg of candesartan and 10 mg of lercanidipine, (xi) 16 mg of candesartan and 20 mg of lercanidipine, (xii) 16 mg of candesartan and 40 mg of lercanidipine, (xiii) 16 mg of candesartan and 60 mg of lercanidipine, (xiv) 16 mg of candesartan and 80 mg of lercanidipine, (xv) 32 mg of candesartan and 2.5 mg of lercanidipine, (xvi) 32 mg of candesartan and 5 mg of lercanidipine, (xvii) 32 mg of candesartan and 10 mg of lercanidipine, (xviii) 32 mg candesartan and 20 mg lercanidipine (xix) 32 mg of candesartan and 40 mg of lercanidipine (xx) 32 mg of candesartan and 60 mg of lercanidipine, or (xxi) 32 mg of candesartan and 80 mg of lercanidipine.

The preferred combinations of telmisartan are: (i) 20 mg of telmisartan and 2.5 mg of lercanidipine, (ii) 20 mg of telmisartan and 5 mg of lercanidipine, (iii) 20 mg of telmisartan and 10 mg of lercanidipine, (iv) 20 mg of telmisartan and 20 mg of lercanidipine (v) 20 mg of telmisartan and 40 mg of lercanidipine, (vi) 20 mg of telmisartan and 60 mg of lercanidipine, (vii) 20 mg of telmisartan and 80 mg of lercanidipine, (viii) 40 mg of telmisartan and 2.5 mg of lercanidipine, (ix) 40 mg of telmisartan and 5 mg of lercanidipine, (x) 40 mg of telmisartan and 10 mg of lercanidipine, (xi) 40 mg of telmisartan and 20 mg of lercanidipine, (xii) 40 mg of telmisartan and 40 mg of lercanidipine, (xiii) 40 mg of telmisartan and 60 mg of lercanidipine, (xiv) 40 mg of telmisartan and 80 mg of lercanidipine, (xv) 80 mg of telmisartan and 2.5 mg of lercanidipine, (xvi) 80 mg of telmisartan and 5 mg of lercanidipine, (xvii) 80 mg of telmisartan and 10 mg of lercanidipine, (xviii) 80 mg telmisartan and 20 mg lercanidipine, (xix) 80 mg of telmisartan and 40 mg of lercanidipine, (xx) 80 mg of telmisartan and 60 mg of lercanidipine, or (xxi) 80 mg of telmisartan and 80 mg of lercanidipine.

The preferred combinations of eprosartan are: (i) 400 mg of eprosartan and 2.5 mg of lercanidipine, (ii) 400 mg of eprosartan and 5 mg of lercanidipine, (iii) 400 mg of eprosartan and 10 mg of lercanidipine, (iv) 400 mg of eprosartan and 20 mg of lercanidipine, (v) 400 mg of eprosartan and 40 mg of lercanidipine, (vi) 400 mg of eprosartan and 60 mg of lercanidipine, (vii) 400 mg of eprosartan and 80 mg of lercanidipine, (viii) 600 mg of eprosartan and 2.5 mg of lercanidipine, (ix) 600 mg of eprosartan and 5 mg of lercanidipine, (x) 600 mg of eprosartan and 10 mg of lercanidipine, (xi) 600 mg of eprosartan and 20 mg of lercanidipine, (xii) 600 mg of eprosartan and 40 mg of lercanidipine, (xiii) 600 mg of eprosartan and 60 mg of lercanidipine, (xiv) 600 mg of eprosartan and 80 mg of lercanidipine, (xv) 800 mg of eprosartan and 2.5 mg of lercanidipine, (xvi) 800 mg of eprosartan and 5 mg of lercanidipine, (xvii) 800 mg of eprosartan and 10 mg of lercanidipine, (xviii) 800 mg eprosartan and 20 mg lercanidipine, (xix) 800 mg of eprosartan and 40 mg of lercanidipine, (xx) 800 mg of eprosartan and 60 mg of lercanidipine, or (xxi) 800 mg of eprosartan and 80 mg of lercanidipine.

Amounts may need to be optimized according to the needs of particular patient subpopulations depending on whether they are responders, partial responders, nonresponders, or naive to lercanidipine and/or ARB monotherapy at a tolerated dose.

In addition, the doses of diuretics that can be added with will be at or below standard doses used for the treatment of hypertension. For example, for HCT, 25-100 mg/day is typically administered. For example, as stated above, in combination with candesartan cilexetil and telmisartan, the oral dose of HCT is 12.5 mg, and may be taken twice daily. Exemplary oral doses for other diuretics are as follows: bendroflumethiazide, about 2.5 to about 20 mg/day; chlorothiazide, about 250 to about 1000 mg/day; chlorthalidone, about 20 to 100 mg once a day, or 100 to 200 mg every other day or once a day for 3 days out of the week; hydroflumethiazide, about 50-100 mg/day; methyclothiazide, about 2.5 to about 5 mg once a day; metolazone, about 2.5 to about 5 mg once a day; polythiazide, about 2 to about 4 mg once a day; amiloride, about 5 to about 10 mg once a day; spironolactone, initially about 50-100 mg once a day, gradually increasing up to about 200 mg/day; triamterene, about 100 mg two times a day; bumetanide, 0.5 to about 2 mg/day; ethacrynic acid, about 50 to about 200 mg/day; and furosemide, about 40 mg two times a day.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents, based upon 100% total weight of the dosage.

The exact dosage and administration regimen utilizing the combination therapies of the present invention is selected in accordance with a variety of factors including type, species, age, weight, sex and medical condition of the patient; the severity and etiology of the hypertension to be treated; the route of administration; the renal and hepatic function of the patient; the treatment history of the patient; and the responsiveness of the patient. Optimal precision in achieving concentrations of active agents within the range that yields efficacy without toxicity requires a regimen based on the kinetics of the drug's availability to target sites. This involves a consideration of the absorption, distribution, metabolism, excretion of a drug, and responsiveness of the patient to the dosage regimen.

However, such fine tuning of the therapeutic regimen is routine in light of the guidelines given herein.

The pharmaceutical composition or unit dosage form may be administered in a single daily dose, or the total daily dosage may be administered in divided doses or using modified release dosage forms (e.g., sustained release, once-a-day dosage forms, etc.). Single daily doses are preferred. In addition, co administration or sequential administration of other active agents may be desirable. For example, especially the addition of a diuretic, but also a preceptor blocker, or an ACE inhibitor to the combination of lercanidipine and ARB is contemplated by the present invention. The dosage amounts of the active agents may be adjusted when combined with other active agents to achieve desired effects (e.g., reduction of blood pressure by a predetermined increment, reduction or avoidance of a particular side-effect).

For combination treatment with lercanidipine and ARB where the active agents are in separate dosage formulations, the active agents can be administered concurrently, or they each can be administered at separately staggered times. For example, the ARB may be administered in the morning and lercanidipine may be administered in the evening, or vice versa. Additional active agents also may be administered simultaneously with either lercanidipine or the ARB at specific intervals from one or the other administration. The order of administration will depend upon the variety of factors including age, weight, sex and medical condition of the patient; the severity and etiology of the hypertension to be treated; the route of administration; the renal and hepatic function of the patient; the treatment history of the patient; and the responsiveness of the patient. Determination of the order of administration may be fine tuned and such fine tuning is routine in light of the guidelines given herein.

In a preferred embodiment of the present invention, the composition is administered daily to the patient. In a further embodiment, the composition of lercanidipine and ARB is formulated into a single dosage form, preferably for administration once daily.

Patients that may be administered the composition described herein include, without limitation, partial responders or nonresponders to monotherapy with an ARB or lercanidipine or with another calcium antagonist or ARB and partial responders and nonresponders to other combination therapies. Another class of patients include responders to monotherapy that suffer from dosage-related side-effects, and responders to monotherapy who have been previously determined (or are expected) to become partial responders or nonresponders over time. The classification of patients into nonresponders, partial responders, and responders to a particular antihypertensive regimen is conventionally made by trial and error.

Recently, pharmacogenomic methods involving haplotyping have been utilized to identify responder patients, a priori, e.g., U.S. Pat. Nos. 6,200,754; 6,183,958; 6,110,684; and WO 98/45477.

### Uses-Methods for Treating Hypertension

The present invention contemplates a method of treating hypertension by administering to a patient a combination of lercanidipine and an ARB, and optionally, a diuretic. In one preferred embodiment, the combination of the two active agents is formulated in one pharmaceutical composition. The patient is administered the combination at prescribed intervals (usually once daily) to maintain a physiologically effective amount of the active agents within the patient's system to produce the desired effect (i.e., a reduction of the patient's blood pressure by the predetermined increment). The composition may be administered by any route, as described above but oral administration is preferred for chronic treatment. The method may be used to treat hypertension in responders, partial responders, and nonresponders of monotherapy.

In addition, the combination of lercanidipine and an ARB, and optionally, a diuretic, can be used to treat a sub-population of hypertensive patients who have tachycardia. It has unexpectedly been shown that the combination of an ARB and lercanidipine has the ability to lower the heart rate relative to lercanidipine alone.

A third use for the combination of the present invention is to prevent or reduce renal failure associated with long-term diuretic use. Preclinical studies demonstrated the protection of lercanidipine in hypertension-induced renal damages. Unlike the conventional type of calcium antagonists that dilate only afferent vessels, lercanidipine dilates both afferent and efferent glomerular arterioles. Sabbatini et al (Sabbatini et al., Hypertension 2000, 35:775-779) demonstrated the histological dilatation of efferent as well as afferent arteriolar lumen by 12-week treatment with lercanidipine in spontaneously hypertensive rats (SHR). Hayashi et al. (in Epstein M.: Calcium antagonist in clinical medicine, ed. 3, New York, Hanley & Belfus, 2001, pp 559-578) demonstrated that 8-week-treatment with lercanidipine prevents the progression of renal injury in subtotally nephrectomized SHR. Moreover, lercanidipine ameliorated the histopathological changes due to hypertension (i.e. glomerular and tubular injury), serum creatinine levels and reduced proteinuria. Thus, the renal protective effects of lercanidipine may be associated with the glomerular hemodynamic action of this agent, i.e. the reduction of glomerular capillary pressure. ARBs are documented to elicit predominant vasodilation of the efferent arteriole, ameliorate glomerular hypertension and afford renal protection (Ichikawa I, Kidney Int, 1996;50:684-692). The combination of lercanidipine and ARB, and optionally a diuretic, present the potential for synergistic renal protection.

The double or triple combination of lercanidipine, ARB and diuretic is also contemplated for the treatment of other pathologies associated with hypertension, such as damage to retinal vasculature which results in glaucoma, and dementia and other cerebrovascular diseases which arise from arterial hypertension.

The neuroprotective effects of lercanidipine on hypertensive retinas of spontaneously hypertensive rats (SHR) has been studied by Sabbatini et al. (Clin Exp Hypertens 2002; 24: 727-40). In SHR, morphological changes, including reduced thickness of the retina and of the inner plexiform, outer nuclear layer, and inner and outer segments plus the outer limiting layer, was observed. In addition, loss of ganglionic neurons was observed, along with GFAP-immunoreactive astrocyte hypertrophy. Lercanidipine and other dihydropyridine CCBs counteracted these phenomena.

In addition, hypertensive elderly patients demonstrated significant improvement of Minimental Status Test after six months of blood pressure control with lercanidipine (Roma et al., J. Hypertension 2002; 20(Suppl. 4): S391), and induced an improvement in the cognitive profile of patients in a Primary Healthcare setting (Tisaire et al., J. Hypertension 2002; 20(Suppl.4): S399). Similarly, the ARB losartan improved cognitive performance after 26 months in a cohort of 69 elderly patients (Tedesco et al., Am. J. Hypertens. 1999; 12: 1130-34).

Another study in SHR demonstrated that lercanidipine, and to a lesser extent hydralazine, was more effective in manidipine and nimodipine in restoring the wall-to-lumen ratio value and other hypertension-associated cerebrovascular changes (Sabbatini et al., Mech. Aging Dev. 2001; 122: 297-307). Candesartan also improved the incidence of major cardiovascular events in elderly patients and reduction in non-fatal stroke (Lithella et al., J. Hypertension 2003; 21: 875-86).

Accordingly, the double or triple combination of lercanidipine with an ARB and, optionally, a diuretic, is anticipated to be superior to the other CCBs for the treatment of these disorders.

### EXAMPLES

The present invention will be better understood by reference to the following Examples, which are provided as exemplary of the invention, and not by way of limitation.

### EXAMPLE I

### Model of Acute Angiotensin-Mediated Renal Hypertension in Anesthetized Rats

### Methods

Male Sprague-Dawley rats, weighing 250-300 g, were anaesthetized with pentobarbital sodium (35 mg/kg, i.p.) and placed on a thermic blanket. The temperature was maintained at 37° C. with a thermoregulator via a rectal probe. The animals were tracheotomized to facilitate spontaneous breathing. A polyethylene catheter was placed in the left jugular vein to allow for infusion of pentobarbital sodium to maintain anesthesia. The left femoral vein and artery were cannulated with polyethylene catheters to allow drugs administration and to monitor blood pressure, respectively.

Animals then underwent a left nephrectomy by excising the left kidney via a flank incision. The right kidney and renal vein, artery and ureter were then exposed via a right retroperitoneal incision, under a dissecting microscope. Silk threads were placed around both vessels and ureter. The cavity was then covered with Vaseline oil. See Recordati, et al. 2000, J. Hypertension, 18:1277-1287.

After 30-60 minutes of basal recordings of arterial blood pressure and heart rate the threads around the renal vessels and ureter were tied close to the renal hilum to induce complete renal ischemia of the right kidney. After 2 hours of ischemia, the threads were removed to allow renal reperfusion and urine output. The reopening of the renal hilum and restoration of renal circulation, induced an increase in blood pressure that peaked at 5-10 min and lasted about 60 min.

For irbesartan experiments and comparisons, control (vehicle) or drug (irbesartan (100 μg/kg), lercanidipine (7.5 μg/kg), or both lercanidipine (7.5 μg/kg) and irbesartan (100 μg/kg)) were administered intravenously at 5 min after reperfusion was begun.

For olmesartan experiments and comparisons, due to the intravenous mode of administration in the animal model, olmesartan free acid was used in these treatments. Control (vehicle) or drug (olmesartan free acid (10 μg/kg), lercanidipine (7.5 μg/kg), or both lercanidipine (7.5 μg/kg) and olmesartan free acid (10 μg/kg)) were administered intravenously at 5 min after reperfusion was begun.

For valsartan experiments and comparisons, drugs (vehicle, valsartan 30 μg/kg), lercanidipine (10 μg/kg), or both lercanidipine (10 μg/kg) and valsartan (30 μg/kg) are administered intravenously at 5 minutes after reperfusion is begun.

Results were expressed as mean values ±S.E. (in the tables) and in percent change observed.

To evaluate the effects of drugs administration on blood pressure and heart rate within each group, two-way ANOVA and Dunnet's test were used (“within treatment”). To evaluate the statistical differences among the treatment groups, data are analyzed using a three-way ANOVA (analysis of variance) with repeated measures on factor time and pre-planned multiple comparisons. The analysis was performed using, for each treatment, delta values (log transformation). Statistical analysis was performed by means of general linear model procedure (GLM) with SAS software version 6.12. (“between treatments”).

### Results

Effects of Treatments on BP

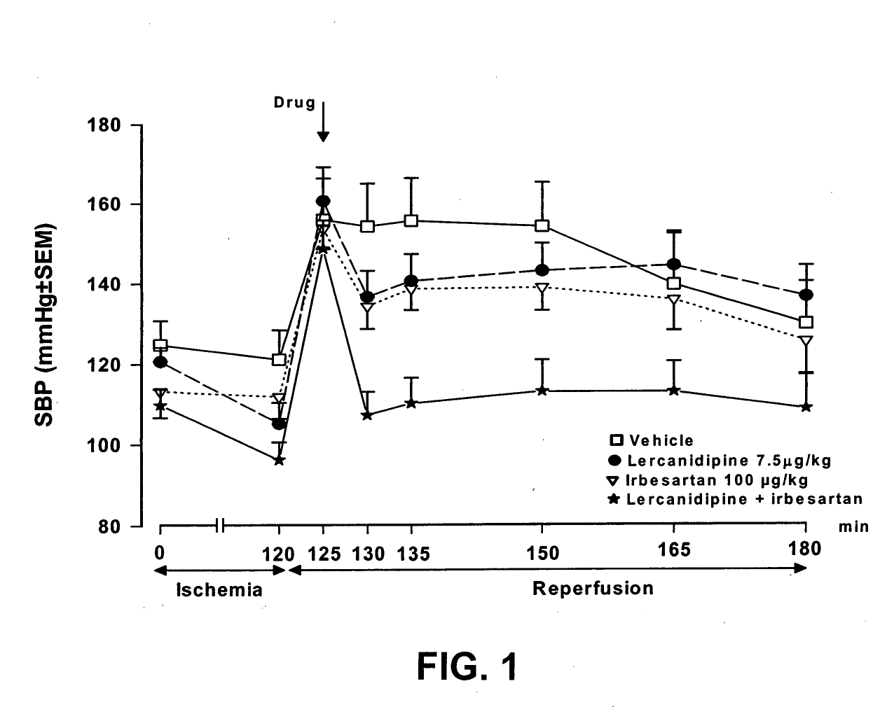

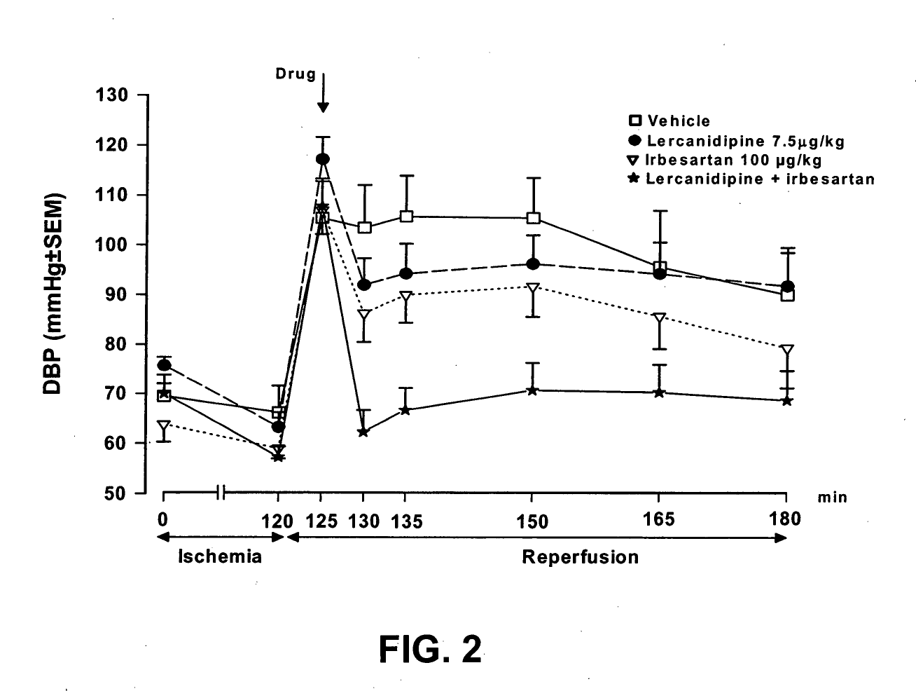

Following reperfusion, a marked and fast increase of blood pressure was observed that had its peak at 5 min after the reopening of vascular renal hilum. In order to compare the effects of single drugs and their combination on the reperfusion-induced hypertension, the compounds were intravenously administered at the peak of induced hypertension (5 min) and statistical analysis (“within groups”) was performed from the time of drug or vehicle administration to the end of experiment (125-180 min). (Table 1; FIGS. 1 and 2).

Lercanidipine and irbesartan. In the control vehicle-treated group, the hypertensive state was maintained without any statistically significant decrease in systolic and diastolic blood pressure up to 25 and 40 min, respectively, after kidney reperfusion When administered alone, both lercanidipine and irbesartan induced a statistically significant decrease in both systolic and diastolic blood pressure (Tables 2 and 3; FIGS. 1 and 2). The observed antihypertensive effect was similar for both drugs. The combination of lercanidipine and irbesartan administered together induced a rapid and significant fall in blood pressure, immediately reversing the ischemia-induced hypertension and lowering blood pressure to normotensive pre-ischemic levels (Table 4; FIGS. 1 and 2).

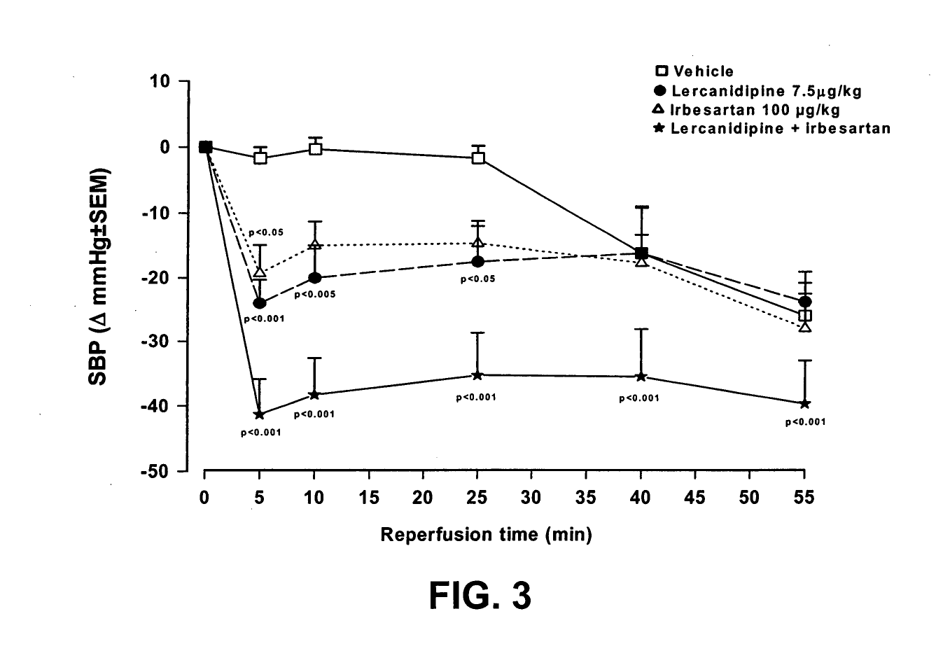

The combination of lercanidipine and irbesartan induced changes in SBP statistically different from those observed after injection of vehicle and irbesartan alone (Table 5). Furthermore, the decrease of SBP induced by the administration of the combination was long-lasting, as the decrease of SBP was significantly different from the vehicle treated group for the whole period of observation (FIG. 3).

The decrease of DBP induced by administration of the two drugs tested alone was significantly different (on the whole) with respect to the vehicle, and the effect observed after administration of the combination was significantly different from all the other treatments (Table 6).

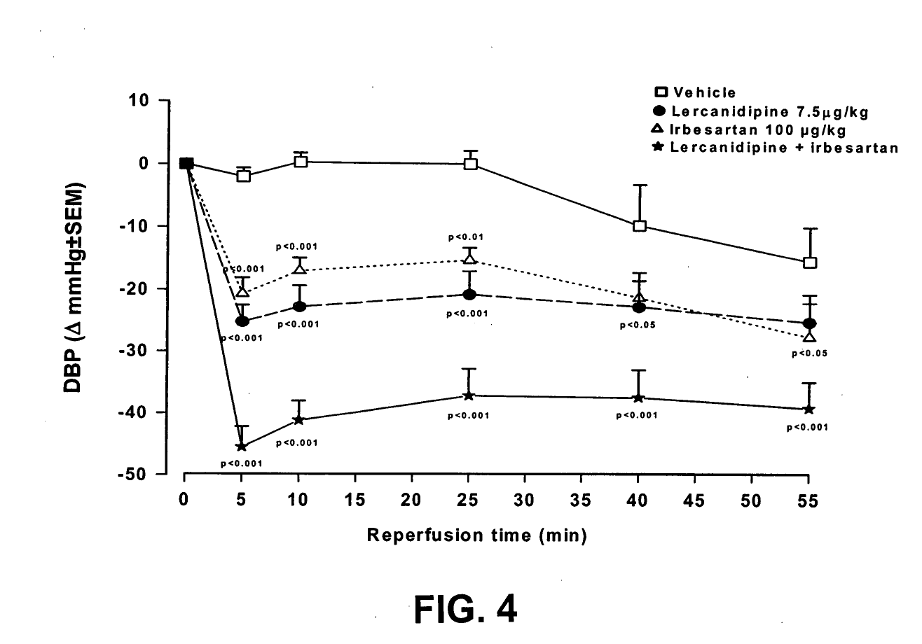

The antihypertensive effect induced by the combination of drugs on diastolic blood pressure was higher than each single drug and statistically different from control group from 5 min to the end of the experiment (FIG. 4).

The data indicate an augmentation of the antihypertensive effect induced by the combination, since the decrease in DBP induced by the drugs in combination was equal or higher than the sum of the decrease induced by each single drug.

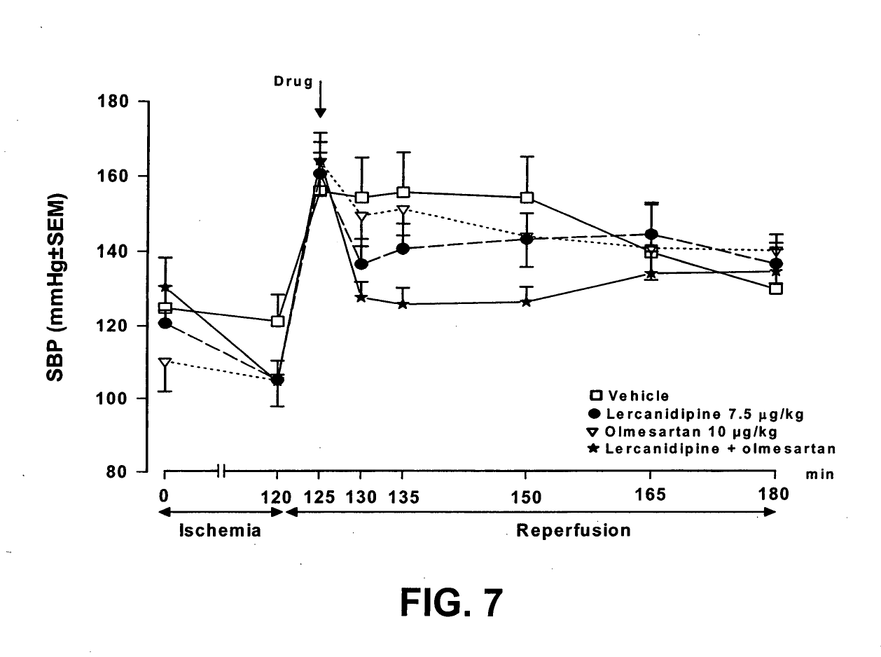

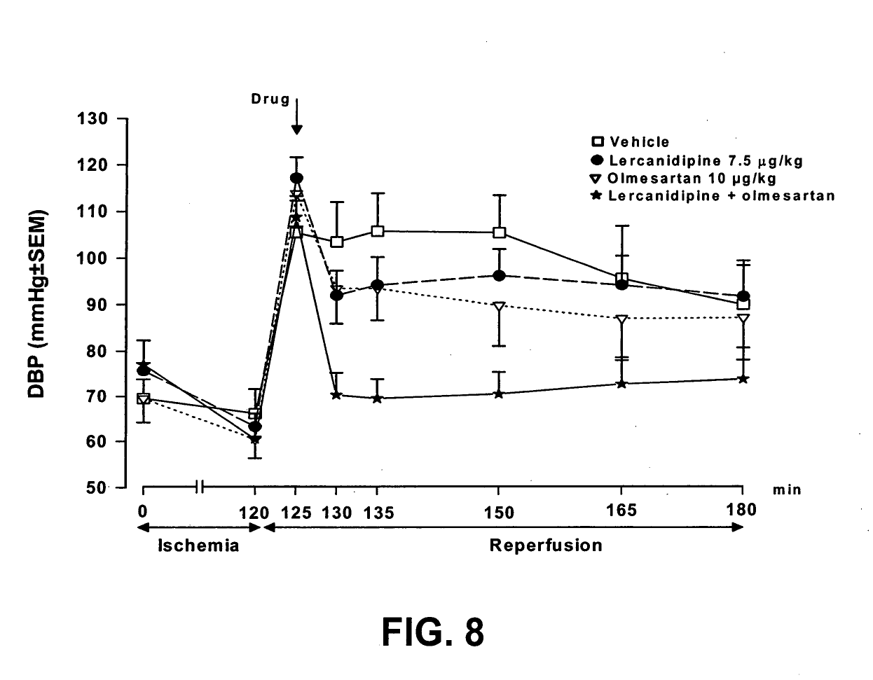

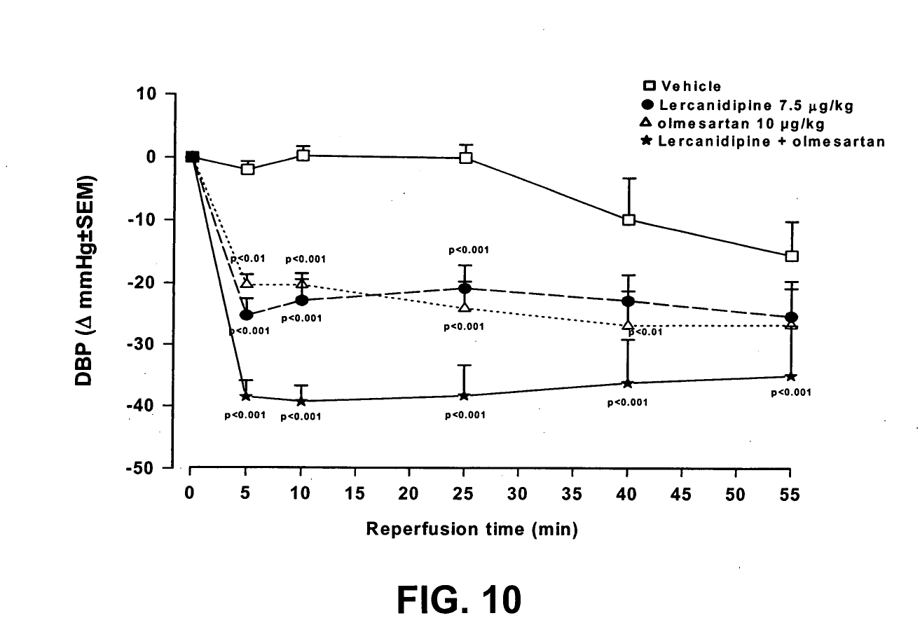

Lercanidipine and olmesartan. In the control vehicle-treated group, the hypertensive state was maintained without statistically significant decrease in diastolic blood pressure up to 40 min (and in systolic blood pressure up to 25 min) after the kidney reperfusion. Table 1; FIGS. 7 and 8. Lercanidipine administration induced a statistically significant decrease in diastolic blood pressure from 5 to 55 min after administration. Table 8; FIGS. 8 and 10.

Olmesartan also induced a statistically significant decrease in diastolic blood pressure from 5 to 55 min after administration, whereas the effects on systolic blood pressure were statistically significant at 40 and 55 min after administration Table 9, FIGS. 7 and 8. The antihypertensive effect of olmesartan alone was similar to that induced by lercanidipine.

The combination of lercanidipine and olmesartan administered together induced a rapid and significant fall in blood pressure, immediately reversing the ischemia-induced hypertension and lowering blood pressure to normotensive pre-ischemic levels. Table 10; FIGS. 7 and 8.

In order to compare the effects of the different treatments on BP, statistical analysis was performed on ΔSBP and ΔDBP values, as described in the Methods Section.

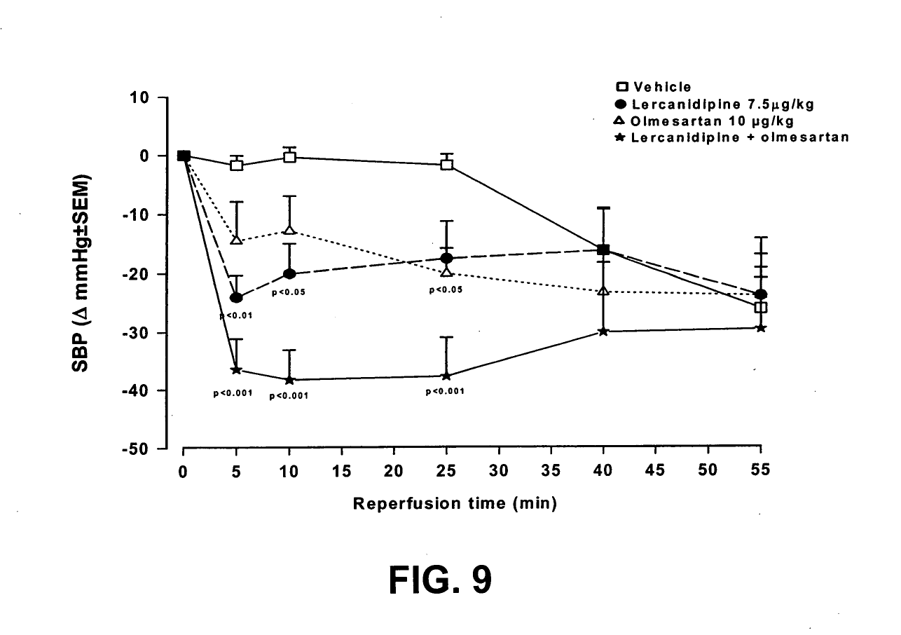

Although when administered to patients, either lercanidipine or olmesartan has significantly lowers hypertension, in these experiments, the changes in SBP induced by injection of lercanidipine and olmesartan alone were not statistically different from those observed in the vehicle-treated animals. The combination of both drugs, however, induced changes statistically different from those observed after injection of vehicle alone (Table 11). The data indicate an augmentation of the antihypertensive effect induced by the lercanidipine-olmesartan combination at 5, 10 min after the administration, since the decrease in SBP was higher or equal than the sum of the decrease induced by each single drug. FIG. 9.

The decrease of DBP induced by administration of lercanidipine and olmesartan tested alone and in combination was significantly different (on the whole) with respect to the vehicle (Table 12). The effect of olmesartan alone was statistically different from control group from 5 to 40 min after administration. The data indicate an augmentation of the antihypertensive effect induced by the combination of lercanidipine and olmesartan on diastolic blood pressure, higher than each drug alone and statistically different from control group from 5 min to the end of the experiment. FIG. 10.

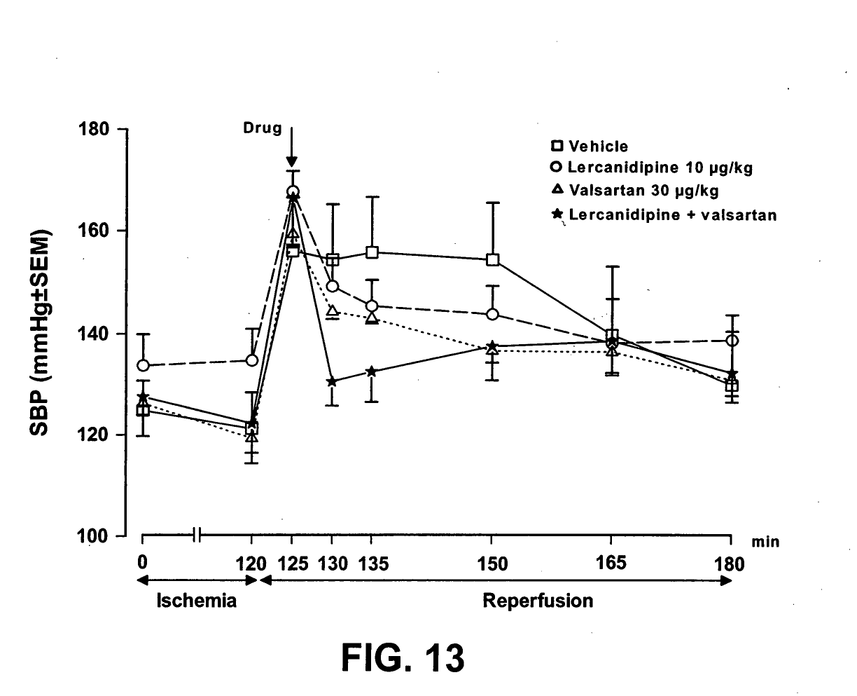

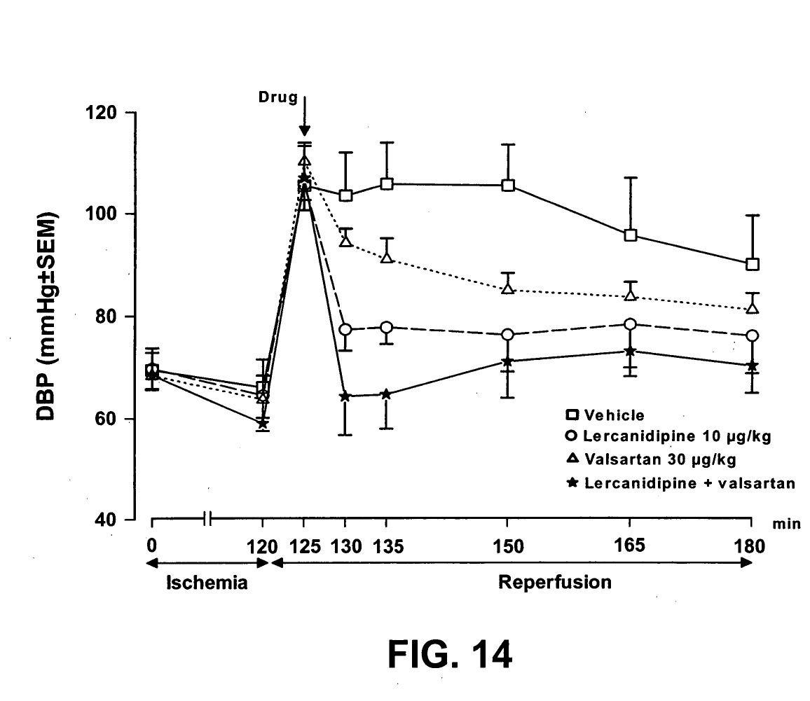

Lercanidipine and valsartan. In the vehicle-treated group, the hypertensive state was maintained without statistically significant decrease in both systolic and diastolic blood pressure up to up to 25 and 40 min, respectively after the kidney reperfusion (Tab. 1; FIGS. 13 and 14); lercanidipine (10 μg/kg) induced a statistically significant decrease in diastolic blood pressure form 5 to 55 min after administration and systolic blood pressure from 10 to 55 min (Table 14; FIGS. 13 and 14).

Valsartan at 30 μg/kg, induced a statistically significant decrease in diastolic blood pressure from 5 to 55 min after administration, whereas the effects on systolic blood pressure were statistically significant at 10 and 55 after administration (Table 15; FIGS. 13 and 14). The antihypertensive effect of valsartan was similar to that induced by lercanidipine.

When lercanidipine and valsartan were administered together (10 μg/kg+30 μg/kg, respectively) they induced a rapid and significant fall in blood pressure, reversing immediately the ischemia-induced hypertension and lowering blood pressure to normotensive pre-ischemic levels (Table 16; FIGS. 13 and 14). The observed effects, however, were not as great as the effects achieved with the lercanidipine/olmesartan and lercanidipine/irbesartan combinations.

In order to compare the effects of the different treatments on BP, statistical analysis was performed on A SBP and A DBP values, as described in the Methods Section.

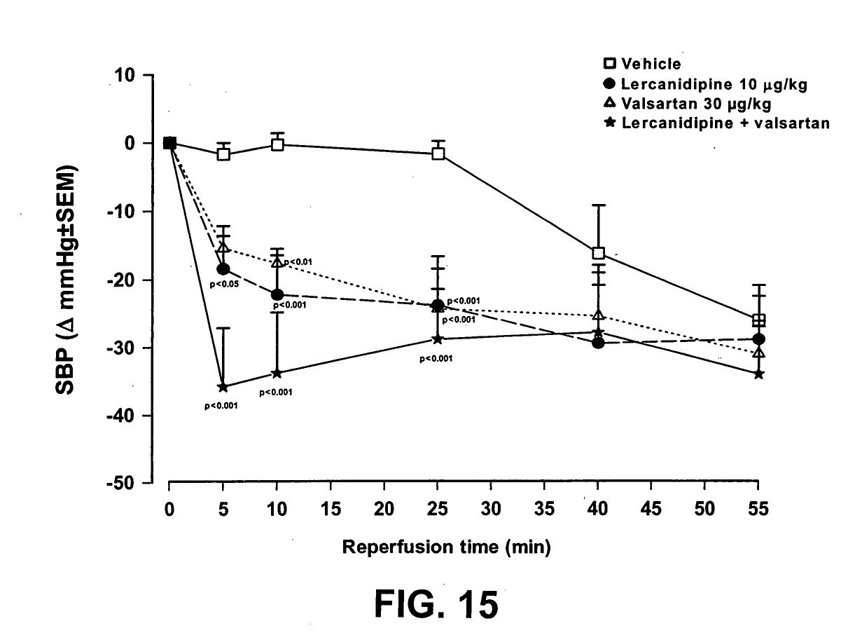

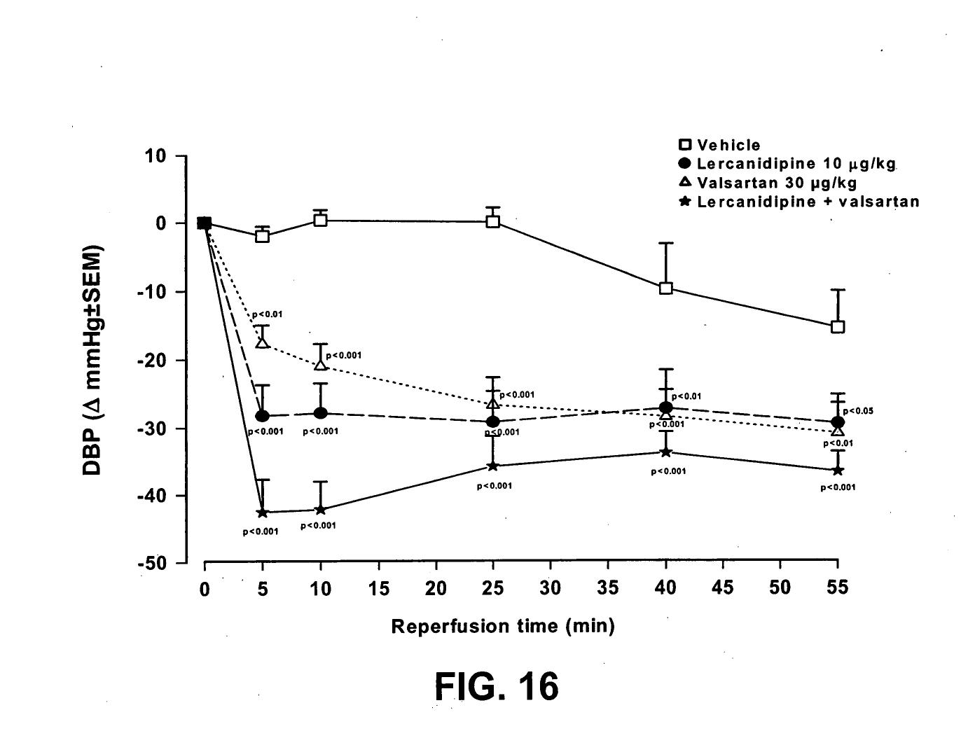

On the whole, the changes in SBP induced by injection of lercanidipine and valsartan alone were not statistically different from those observed in the vehicle-treated animals, whereas the combination of both drugs induced changes statistically different from those observed after injection of vehicle alone from 5 to 25 min after administration (Table 17). The data indicate an augmentation of the antihypertensive effect induced by the combination at 5 min after the administration, since the decrease in SBP was higher than the sum of the decrease induced by each single drug, as shown in FIG. 15. The decrease of DBP induced by administration of the two drugs tested alone and in combination was significantly different (on the whole) with respect to the vehicle (Table 18). The antihypertensive effect induced by the combination of drugs on diastolic blood pressure was higher than each single drug and statistically different from control group from 5 min to the end of the experiment (FIG. 16).

Effects of Treatments on HR

Lercanidipine and irbesartan. Treatment with irbesartan alone induced no statistically significant change in heart rate. Treatment with lercanidipine alone, however, was characterized by an increase in heart rate. The increase in heart rate observed in this animal model was probably due to the baroreflex-activation, that was more marked than that observed in the vehicle group. Interestingly, when lercanidipine was administered together with irbesartan the observed change in heart rate was not significantly different from the changes heart rate observed in the vehicle- and irbesartan-treated groups (Table 7).

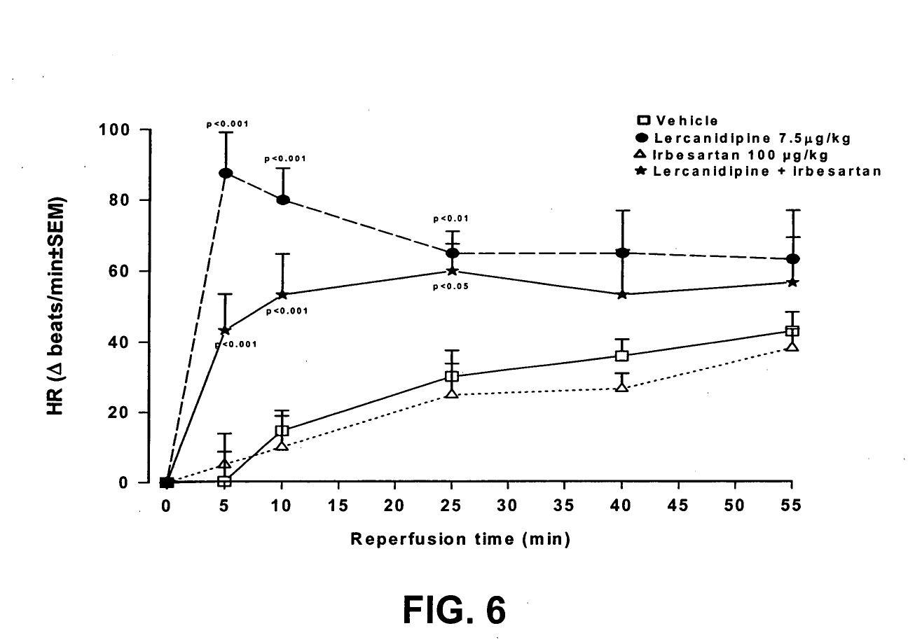

The combination of lercanidipine and irbesartan reduced the tachycardic effect observed in this animal model following administration of lercanidipine alone within 20 min after administration (FIG. 6). The increase in HR observed 5 min after administration of lercanidipine alone and of the combination, in fact, were significantly different (p<0.05).

The results from the present study demonstrate that the combination of lercanidipine and the ARB irbesartan immediately reduced, in an augmented manner, the extent of renal hypertension for the entire duration of the experiment. The effects of combination were longer lasting in comparison with the effects of the compounds administered alone, in particular with regard to the decrease of SBP (FIG. 3).

Furthermore, the combination of lercanidipine and irbesartan reduced the tachycardic effect observed following administration of lercanidipine alone to this animal model.

TABLE 1
Observed effects on systolic blood pressure (SBP),
diastolic blood pressure (DBP) and heart rate (HR)
following intravenous administration of vehicle
(1 ml/kg) in uninephrectomized anesthetized rats.
Mean value ± S.E.M. (n = 6).

| Time | SBP | DBP | HR |
|---|---|---|---|
| min | mmHg | mmHg | beats/min |
| 0 | 124.8 ± 6.0 | 69.5 ± 4.3 | 369.7 ± 13.5 |
| Ischemia | | | |
| 120 | 121.2 ± 7.3 | 66.2 ± 5.4 | 357.7 ± 10.4 |
| Reperfusion | | | |
| 125 | 156.0 ± 10.4 | 105.5 ± 7.8 | 338.5 ± 4.7 |
| Vehicle | | | |
| 130 | 154.3 ± 10.8 | 103.5 ± 8.5 | 338.8 ± 10.7 |
| 135 | 155.7 ± 10.8 | 105.8 ± 8.1 | 353.2 ± 8.1 |
| 150 | 154.3 ± 11.1 | 105.5 ± 8.0 | 368.7 ± 11.4 b |
| 165 | 139.8 ± 13.2 b | 95.7 ± 11.3 | 374.5 ± 6.4 b |
| 180 | 130.0 ± 10.5 b | 90.0 ± 9.6 b | 381.5 ± 7.5 b |

b = p < 0.01 v 125 min (within treatment) (Two way ANOVA and Dunnett's test)

TABLE 2
Observed effects on systolic blood pressure (SBP),
diastolic blood pressure (DBP) and heart rate (HR)
following intravenous administration of lercanidipine
(7.5 μg/kg) in uninephrectomized anesthetized rats.
Mean value ± S.E.M. (n = 6)..

| Time | SBP | DBP | HR |
|---|---|---|---|
| min | mmHg | mmHg | beats/min |
| 0 | 120.7 ± 3.5 | 75.7 ± 1.7 | 383.3 ± 12.0 |
| Ischemia | | | |
| 120 | 105.2 ± 5.3 | 63.3 ± 2.9 | 381.7 ± 7.5 |
| Reperfusion | | | |
| 125 | 160.7 ± 8.5 | 117.2 ± 4.4 | 346.7 ± 8.4 |
| Drug | | | |
| 130 | 136.7 ± 7.4 | b 92.0 ± 5.4 b | 434.2 ± 15.3 b |
| 135 | 140.7 ± 7.1 | b 94.3 ± 6.0 b | 426.7 ± 14.3 b |
| 150 | 143.2 ± 7.2 | b 96.3 ± 5.7 b | 411.7 ± 12.8 b |
| 165 | 144.5 ± 9.1 | b 94.3 ± 6.3 b | 411.7 ± 15.6 b |
| 180 | 136.8 ± 8.4 | b 91.8 ± 6.7 b | 410.0 ± 18.3 b |

b = p < 0.01 vs 125 min (within treatment). (Two way ANOVA and Dunnett's test)

TABLE 3
Observed effects on systolic blood pressure (SBP),
diastolic blood pressure (DBP) and heart rate (HR) following
intravenous administration of irbesartan (100 μg/kg)
in uninephrectomized anesthetized rats.
Mean value ± S.E.M. (n = 6).

| Time | SBP | DBP | HR |
|---|---|---|---|
| min | mmHg | mmHg | beats/min |
| 0 | 113.3 ± 6.7 | 63.8 ± 3.5 | 373.3 ± 14.5 |
| Ischemia | | | |
| 120 | 112.0 ± 5.6 | 59.0 ± 2.0 | 360.0 ± 13.4 |
| Reperfusion | | | |
| 125 | 153.7 ± 5.1 | 107.0 ± 4.8 | 336.7 ± 14.1 |
| Drug | | | |
| 130 | 134.3 ± 5.5 b | 86.3 ± 5.8 b | 341.7 ± 13.8 |
| 135 | 138.7 ± 5.3 b | 90.0 ± 5.6 b | 346.7 ± 13.6 |
| 150 | 139.0 ± 5.7 b | 91.7 ± 6.1 b | 361.7 ± 7.5 a |
| 165 | 136.0 ± 7.6 b | 85.7 ± 6.6 b | 363.3 ± 11.8 a |
| 180 | 125.7 ± 8.3 | b 79.3 ± 8.1 b | 375.0 ± 13.1 b |

a = p < 0.05;
b = p < 0.01 v 125 min (within treatment). (Two way ANOVA and Dunnett's test)

TABLE 4
Observed effects on systolic blood pressure (SBP),
diastolic blood pressure (DBP) and heart rate (HR)
following intravenous administration of lercanidipine
(7.5 μg/kg) and irbesartan (100 μg/kg) in
uninephrectomized anesthetized rats.
Mean value ± S.E.M. (n = 6).

| Time | SBP | DBP | HR |
|---|---|---|---|
| min | mmHg | mmHg | beats/min |
| 0 | 109.8 ± 4.0 | 70.0 ± 2.0 | 388.3 ± 4.0 |
| Ischemia | | | |
| 120 | 96.3 ± 4.3 | 57.3 ± 2.1 | 368.3 ± 8.3 |
| Reperfusion | | | |
| 125 | 148.7 ± 7.1 | 107.8 ± 5.0 | 346.7 ± 9.5 |
| Drug | | | |
| 130 | 107.3 ± 5.9 b | 62.3 ± 4.4 b | 390.0 ± 10.3 b |
| 135 | 110.3 ± 6.3 b | 66.7 ± 4.5 b | 400.0 ± 8.9 b |
| 150 | 113.3 ± 7.8 b | 70.7 ± 5.6 b | 406.7 ± 6.7 b |
| 165 | 113.2 ± 7.5 b | 70.3 ± 5.6 b | 400.0 ± 7.3 b |
| 180 | 109.0 ± 8.6 b | 68.7 ± 6.0 b | 403.3 ± 8.0 b |

b = p < 0.01 v 125 min (within treatment). (Two way ANOVA and Dunnett's test)

TABLE 5
Systolic blood pressure differences among groups

| Treatments | Vehicle | Irbesartan | Lercanidipine |
|---|---|---|---|
| Vehicle | — | | |
| Irbesartan | ns | — | |
| Lercanidipine | ns | ns | — |
| Ler + Irbes | p < 0.01 | p < 0.05 | ns |

TABLE 6
Diastolic blood pressure: general differences among groups

| Treatments | Vehicle | Irbesartan | Lercanidipine |
|---|---|---|---|
| Vehicle | — | | |
| Irbesartan | p < 0.05 | — | |
| Lercanidipine | p < 0.05 | ns | — |
| Ler + Irbes | p < 0.001 | p < 0.005 | p < 0.01 |

TABLE 7
Heart rate differences among groups

| Treatments | Vehicle | Irbesartan | Lercanidipine |
|---|---|---|---|
| Vehicle | — | | |
| Irbesartan | ns | — | |
| Lercanidipine | p < 0.01 | p < 0.001 | — |
| Ler + Irbes | ns | p < 0.05 | ns |

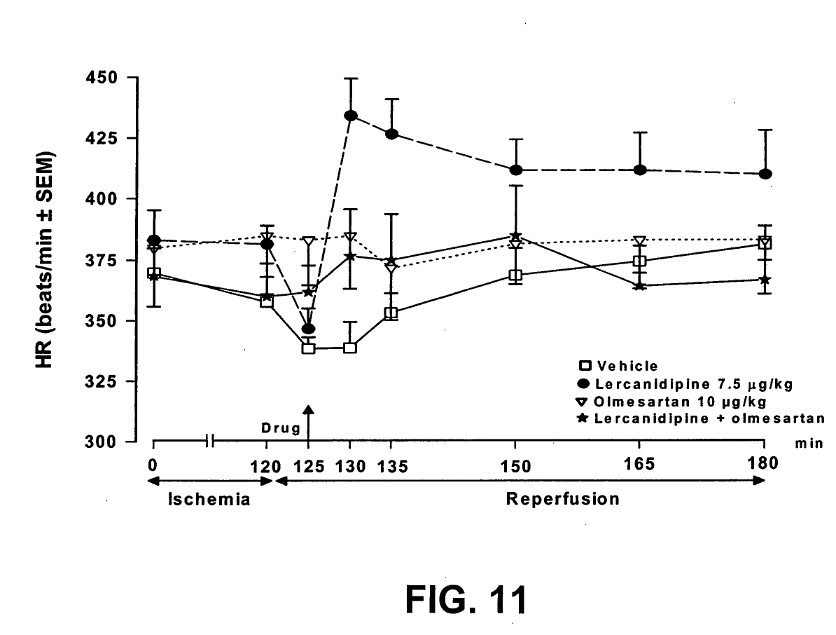

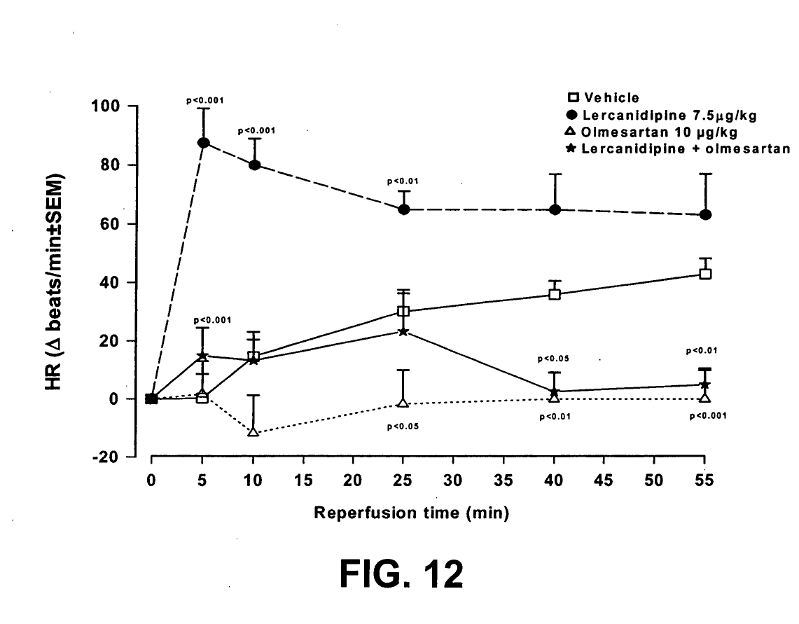

Lercanidipine and olmesartan. Treatment with olmesartan alone induced no statistically significant change in heart rate (Table 9), whereas treatment with lercanidipine alone was characterized by an increase in heart rate. The increase in heart rate observed in this animal model was probably due to the baroreflex-activation, that was more marked than that observed in the vehicle group. Interestingly, when lercanidipine was administered together with olmesartan the observed change in heart rate in the animal model was not significantly different from the changes heart rate observed in the vehicle- and olmesartan-treated groups. Table 13 and FIGS. 11 and 12. Hence, the combination of olmesartan and lercanidipine completely inhibited the tachycardic effect observed following administration of lercanidipine alone. The observed differences in heart rate observed following administration of lercanidipine alone versus administration of the combination of lercanidipine and olmesartan were significant (p<0.01) throughout the observed treatment period.

Observed effects on systolic blood pressure (SBP), diastolic blood pressure (DBP) and heart rate (HR) following intravenous administration of vehicle (1 ml/kg) in uninephrectomized anesthetized rats is as in Table 1, above.

TABLE 8
Observed effects on systolic blood pressure (SBP),
diastolic blood pressure (DBP) and heart rate (HR) following
intravenous administration of lercanidipine
(7.5 μg/kg) in uninephrectomized anesthetized rats.
Mean value ± S.E.M. (n = 6).

| Time | SBP | DBP | HR |
|---|---|---|---|
| min | mmHg | mmHg | beats/min |
| 0 | 120.7 ± 3.5 | 75.7 ± 1.7 | 383.3 ± 12.0 |
| Ischemia | | | |
| 120 | 105.2 ± 5.3 | 63.3 ± 2.9 | 381.7 ± 7.5 |
| Reperfusion | | | |
| 125 | 160.7 ± 8.5 | 117.2 ± 4.4 | 346.7 ± 8.4 |
| Drug | | | |
| 130 | 136.7 ± 7.4 | 92.0 ± 5.4b | 434.2 ± 15.3b |
| 135 | 140.7 ± 7.1b | 94.3 ± 6.0b | 426.7 ± 14.3b |
| 150 | 143.2 ± 7.2 | 96.3 ± 5.7b | 411.7 ± 12.8b |
| 165 | 144.5 ± 9.1 | 94.3 ± 6.3b | 411.7 ± 15.6b |
| 180 | 136.8 ± 8.4b | 91.8 ± 6.7b | 410.0 ± 18.3b |

b = p < 0.01 vs 125 min (within treatment)

TABLE 9
Observed effects on systolic blood pressure (SBP),
diastolic blood pressure (DBP) and heart rate (HR) following
intravenous administration of olmesartan (10 μg/kg)
in uninephrectomized anesthetized rats.
Mean value ± S.E.M. (n = 6).

| Time | SBP | DBP | HR |
|---|---|---|---|
| min | mmHg | mmHg | beats/min |
| 0 | 110.3 ± 8.3 | 69.5 ± 5.3 | 380.0 ± 24.2 |
| Ischemia | | | |
| 120 | 105.0 ± 7.2 | 60.5 ± 4.1 | 385.0 ± 24.0 |
| Reperfusion | | | |
| 125 | 164.0 ± 9.0 | 113.8 ± 7.0 | 383.3 ± 18.7 |
| Drug | | | |
| 130 | 149.5 ± 8.2 | 93.5 ± 7.6 b | 385.0 ± 21.9 |
| 135 | 151.2 ± 7.0 | 93.5 ± 6.9 b | 371.7 ± 21.4 |
| 150 | 144.0 ± 8.0 | 89.8 ± 8.7 b | 381.7 ± 16.8 |
| 165 | 140.8 ± 8.3 b | 87.0 ± 9.0 b | 383.3 ± 20.1 |
| 180 | 140.2 ± 8.7 b | 87.2 ± 9.1 b | 383.3 ± 22.3 |

b = p < 0.01 v 125 min (within treatment).

TABLE 10
Observed effects on systolic blood pressure (SBP),
diastolic blood pressure (DBP) and heart rate (HR)
following intravenous administration of lercanidipine
(7.5 μg/kg) and olmesartan (10 μg/kg) in
uninephrectomized anesthetized rats.
Mean value ± S.E.M. (n = 6).

| Time | SBP | DBP | HR |
|---|---|---|---|
| min | mmHg | mmHg | beats/min |
| 0 | 130.5 ± 7.9 | 77.0 ± 5.3 | 368.3 ± 11.7 |
| Ischemia | | | |
| 120 | 104.7 ± 1.9 | 60.5 ± 3.0 | 360.0 ± 13.7 |
| Reperfusion | | | |
| 125 | 164.2 ± 7.5 | 108.8 ± 3.5 | 361.7 ± 11.1 |
| Drug | | | |
| 130 | 127.7 ± 4.3 b | 70.3 ± 4.9 b | 376.7 ± 18.9 |
| 135 | 125.8 ± 4.5 b | 69.5 ± 4.3 b | 375.0 ± 18.6 |
| 150 | 126.5 ± 4.1 b | 70.5 ± 4.9 b | 385.0 ± 20.3 |
| 165 | 134.2 ± 5.8 b | 72.7 ± 5.9 b | 364.2 ± 5.5 |
| 180 | 134.7 ± 7.5 b | 73.8 ± 7.0 b | 366.7 ± 8.4 |

b = p < 0.01 v 125 min (within treatment).

TABLE 11
Systolic blood pressure differences among treatment groups

| Treatment | Vehicle | Olmesartan | Lercanidipine |
|---|---|---|---|
| Vehicle | — | | |
| Olmesartan | ns | — | |
| Lercanidipine | ns | ns | — |
| Ler + Olmes | p < 0.05 | ns | ns |

TABLE 12
Diastolic blood pressure differences among treatment groups

| Treatment | Vehicle | Olmesartan | Lercanidipine |
|---|---|---|---|
| Vehicle | — | | |
| Olmesartan | p < 0.05 | — | |
| Lercanidipine | p < 0.05 | ns | — |
| Ler + Olmes | p < 0.001 | ns | ns |

TABLE 13
Heart rate differences among treatment groups

| Treatments | Vehicle | Olmesartan | Lercanidipine |
|---|---|---|---|
| Vehicle | — | | |
| Olmesartan | ns | — | |
| Lercanidipine | p < 0.01 | p < 0.001 | — |
| Ler + Olmes | ns | ns | p < 0.001 |

Lercanidipine and valsartan. Valsartan induced no statistically significant modification on heart rate (Table 15), whereas lercanidipine is characterized by a positive chronotropic effect in the animal model, probably due to the baroreflex-activation, that was more marked than that observed in the vehicle group.

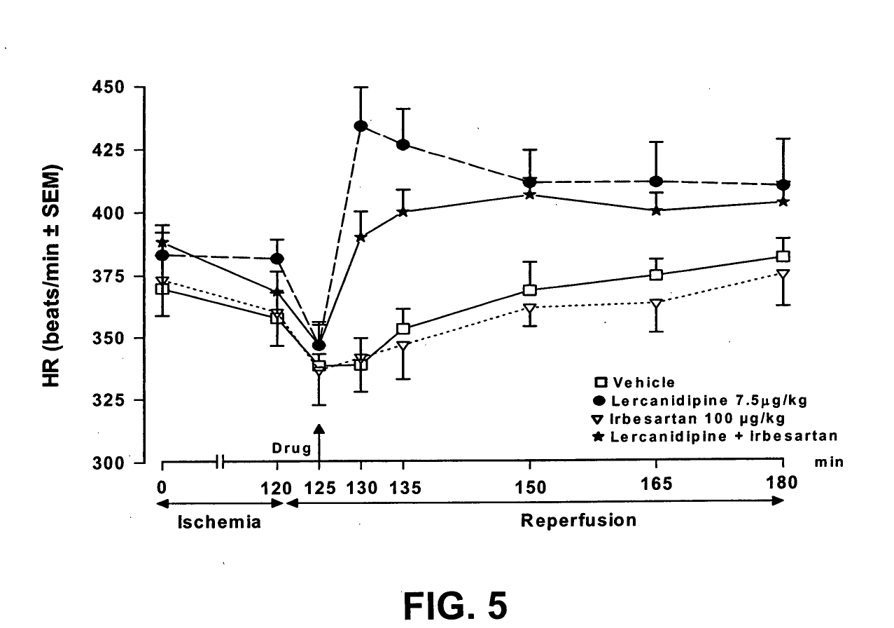

Interestingly, when lercanidipine was administered together with valsartan the changes in heart rate observed in the animal model were not different from those of the vehicle- and valsartan-treated groups and statistically different from lercanidipine group (Table 19): as shown in FIGS. 5 and 6, the combination of lercanidipine and valsartan reduced the tachycardic effect due acute administration of lercanidipine alone.

Observed effects on systolic blood pressure (SBP), diastolic blood pressure (DBP) and heart rate (HR) following intravenous administration of intravenous administration of vehicle (1 ml/kg) in uninephrectomized anesthetized rats, is the same as in Table 1, above.

TABLE 14
Observed effects on systolic blood pressure (SBP),
diastolic blood pressure (DBP) and heart rate (HR)
following intravenous administration of lercanidipine
(10 μg/kg) in uninephrectomized anesthetized rats.
Mean value ± S.E.M. (n = 5).

| Time | SBP | DBP | HR |
|---|---|---|---|
| min | mmHg | mmHg | beats/min |
| 0 | 133.8 ± 6.1 | 69.8 ± 4.1 | 350.0 ± 3.2 |
| Ischemia | | | |
| 120 | 134.8 ± 6.2 | 64.6 ± 4.5 | 350.0 ± 3.5 |
| Reperfusion | | | |
| 125 | 167.6 ± 4.0 | 105.6 ± 5.0 | 345.0 ± 5.5 |
| Drug | | | |
| 130 | 149.2 ± 5.4 | 77.4 ± 4.1 b | 434.0 ± 27.9 b |
| 135 | 145.4 ± 5.0 b | 77.8 ± 3.2 b | 438.0 ± 22.2 b |
| 150 | 143.8 ± 5.5 b | 76.4 ± 7.2 b | 425.0 ± 14.7 b |
| 165 | 138.2 ± 8.6 b | 78.4 ± 8.4 b | 423.0 ± 15.1 b |
| 180 | 138.8 ± 4.9 b | 76.2 ± 7.3 b | 421.0 ± 14.7 b |

b = p < 0.01 vs 125 min (within treatment).

TABLE 15
Observed effects on systolic blood pressure (SBP),
diastolic blood pressure (DBP) and heart rate (HR)
following intravenous administration of valsartan
(30 μg/kg) in uninephrectomized anesthetized rats.
Mean value ± S.E.M. (n = 7).

| Time | SBP | DBP | HR |
|---|---|---|---|
| min | mmHg | mmHg | beats/min |
| 0 | 126.4 ± 5.5 | 66.7 ± 4.1 | 372.9 ± 8.4 |
| Ischemia | | | |
| 120 | 120.1 ± 4.3 | 62.1 ± 4.4 | 366.4 ± 10.9 |
| Reperfusion | | | |
| 125 | 161.0 ± 2.4 | 108.1 ± 3.8 | 362.1 ± 12.9 |
| Drug | | | |
| 130 | 145.6 ± 1.7 | 90.4 ± 4.4 b | 372.1 ± 10.0 |
| 135 | 143.4 ± 1.0 a | 87.1 ± 5.2 b | 381.4 ± 7.3 |
| 150 | 136.7 ± 1.9 b | 81.4 ± 4.5 b | 382.1 ± 6.9 |
| 165 | 135.6 ± 4.0 b | 79.7 ± 4.6 b | 375.7 ± 7.6 |
| 180 | 130.0 ± 3.9 b | 77.3 ± 4.7 b | 368.6 ± 6.7 |

a = p < 0.05;
b = p < 0.01 v 125 min (within treatment).

TABLE 16
Observed effects on systolic blood pressure (SBP),
diastolic blood pressure (DBP) and heart rate (HR)
following intravenous administration of lercanidipine
(10 μg/kg) and valsartan (30 μg/kg) in
uninephrectomized anesthetized rats.
Mean value ± S.E.M. (n = 5).

| Time | SBP | DBP | HR |
|---|---|---|---|
| min | mmHg | mmHg | beats/min |
| 0 | 127.6 ± 3.7 | 68.6 ± 2.7 | 378.0 ± 13.9 |
| Ischemia | | | |
| 120 | 122.2 ± 5.8 | 59.0 ± 1.5 | 358.0 ± 9.7 |
| Reperfusion | | | |
| 125 | 166.4 ± 9.7 | 107.0 ± 4.4 | 350.0 ± 6.3 |
| Drug | | | |
| 130 | 130.6 ± 4.8 b | 64.4 ± 7.7 b | 402.0 ± 20.1 b |
| 135 | 132.6 ± 6.0 b | 64.8 ± 6.9 b | 395.0 ± 14.8 a |
| 150 | 137.6 ± 6.7 b | 71.2 ± 7.1 b | 402.0 ± 18.5 b |
| 165 | 138.6 ± 6.2 b | 73.2 ± 4.9 b | 411.0 ± 18.6 b |
| 180 | 132.4 ± 4.5 b | 70.4 ± 5.3 b | 410.0 ± 16.4 b |

a = p < 0.05;
b = p < 0.01 v 125 min (within treatment).

TABLE 17
Systolic blood pressure: general differences among groups

| Treatments | Vehicle | Valsartan | Lercanidipine |
|---|---|---|---|
| Vehicle | — | | |
| Valsartan | ns | — | |
| Lercanidipine | ns | ns | — |
| Ler + Vals | p < 0.05 | ns | ns |

TABLE 18
Diastolic blood pressure: general differences among groups

| Treatments | Vehicle | Valsartan | Lercanidipine |
|---|---|---|---|
| Vehicle | — | | |
| Valsartan | p < 0.01 | — | |
| Lercanidipine | p < 0.01 | ns | — |
| Ler + Vals | p < 0.001 | ns | ns |

TABLE 19
Heart rate: general differences among groups

| Treatments | Vehicle | Valsartan | Lercanidipine |
|---|---|---|---|
| Vehicle | — | | |
| Valsartan | ns | — | |
| Lercanidipine | p < 0.05 | p < 0.01 | — |
| Ler + Vals | ns | ns | ns |

The effects on hear-rate observed in the animal model following treatment with the lercanidipine alone or lercanidipine in combination with ARBs indicate the combination of lercanidipine and an ARB would be useful in treatment of hypertensive patients who also exhibit tachycardia, either before treatment or as a result of treatment.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

What is claimed:

1. A method for treating hypertension in a patient in need thereof, said method comprising administering to said patient a first amount of an angiotensin II receptor blocker and a second amount of lercanidipine, wherein said amounts in combination are effective to reduce blood pressure in said patient by at least a predetermined increment; wherein each of said first amount and said second amount administered individually is ineffective to produce a reduction in blood pressure in said patient or said reduction in blood pressure is less than said predetermined increment
with the proviso that said angiotensin II receptor blocker is not candesartan.

2. The method of claim 1, which method further comprises administrating a diuretic.

3. The method of claim 1, wherein the angiotensin II receptor blocker is olmesartan medoxomil.

4. The method as defined in claim 3, wherein (i) said first amount is 10 mg and said second amount is 2.5 mg, (ii) said first amount 10 mg and said second amount is 5 mg, (iii) said first amount is 10 mg and said second amount is 10 mg, (iv) said first amount is 10 mg and said second amount is 20 mg, (v) said first amount 10 mg and said second amount is 40 mg, (vi) said first amount 10 mg and said second amount is 60 mg, (vii) said first amount 10 mg and said second amount is 80 mg, (viii) said first amount 20 mg and said second amount is 2.5 mg, (ix) said first amount 20 mg and said second amount is 5 mg, (x) said first amount 20 mg and said second amount is 10 mg, (xi) said first amount 20 mg and said second amount is 20 mg, (xii) said first amount 20 mg and said second amount is 40 mg, (xiii) said first amount 20 mg and said second amount is 60 mg, (xiv) said first amount 20 mg and said second amount is 80 mg, (xv) said first amount 40 mg and said second amount is 2.5 mg, (xvi) said first amount 40 mg and said second amount is 5 mg, (xvii) said first amount 40 mg and said second amount is 10 mg, (xviii) said first amount 40 mg and said second amount is 20 mg, (xix) said first amount 40 mg and said second amount is 40 mg, (xx) said first amount 40 mg and said second amount is 60 mg, or (xxi) said first amount 40 mg of olmesartan and said second amount is 80 mg.

5. The method as defined in claim 4, wherein said first amount is from about 20-40 mg per day and said second amount is from about 10-20 mg/day.

6. The method of claim 2, wherein the diuretic is hydrochlorothiazide or chlorthalidone.

7. The method of claim 5, wherein the hydrochlorothiazide is administered in an amount from about 20-50 mg/day.

8. The method of claim 1, wherein the wherein the angiotensin II receptor blocker is irbesartan.

9. The method of claim 8, wherein (i) said first amount is 75 mg and said second amount is 2.5 mg, (ii) said first amount is 75 mg and said second amount is 5 mg, (iii) said first amount is 75 mg and said second amount is 10 mg, (iv) said first amount is 75 mg and said second amount is 20 mg, (v) said first amount is 75 mg and said second amount is 40 mg, (vi) said first amount is 75 mg and said second amount is 60 mg, (vii) said first amount is 75 mg and said second amount is 80 mg, (viii) said first amount is 150 mg and said second amount is 2.5 mg, (ix) said first amount is 150 mg and said second amount is 5 mg, (x) said first amount is 150 mg and said second amount is 10 mg, (xi) said first amount is 150 mg and said second amount is 20 mg, (xii) said first amount is 150 mg and said second amount is 40 mg, (xiii) said first amount is 150 mg and said second amount is 60 mg, (xiv) said first amount is 150 mg and said second amount is 80 mg, (xv) said first amount is 300 mg and said second amount is 2.5 mg, (xvi) said first amount is 300 mg and said second amount is 5 mg, (xvii) said first amount is 300 mg and said second amount is 10 mg, (xviii) said first amount is 300 mg and said second amount is 20 mg, (xix) said first amount is 300 mg and said second amount is 40 mg, (xx) said first amount is 300 mg and said second amount is 60 mg, or (xxi) said first amount is 300 mg and said second amount is 80 mg.

10. The method of claim 1, wherein said first amount is from about 150-300 mg per day and said second amount is from about 10-20 mg per day.

11. A method for treating hypertension in a nonresponder or partial responder patient in need thereof, said method comprising administering to said patient a first amount of an angiotensin II receptor blocker and a second amount of lercanidipine, wherein said amounts in combination are effective to reduce blood pressure in said patient by at least a predetermined increment, wherein said patient has been previously determined to be a nonresponder or partial responder to monotherapy with lercanidipine or a nonresponder or partial responder to combination therapy involving a different combination of antihypertensive agents,
with the proviso that said angiotensin II receptor blocker is not candesartan.

12. The method of claim 11, which method further comprises administrating a diuretic.

13. The method of claim 11, wherein the angiotensin II receptor blocker is olmesartan medoxomil.

14. The method as defined in claim 13, wherein (i) said first amount is 10 mg and said second amount is 2.5 mg, (ii) said first amount 10 mg and said second amount is 5 mg, (iii) said first amount is 10 mg and said second amount is 10 mg, (iv) said first amount is 10 mg and said second amount is 20 mg, (v) said first amount 10 mg and said second amount is 40 mg, (vi) said first amount 10 mg and said second amount is 60 mg, (vii) said first amount 10 mg and said second amount is 80 mg, (viii) said first amount 20 mg and said second amount is 2.5 mg, (ix) said first amount 20 mg and said second amount is 5 mg, (x) said first amount 20 mg and said second amount is 10 mg, (xi) said first amount 20 mg and said second amount is 20 mg, (xii) said first amount 20 mg and said second amount is 40 mg, (xiii) said first amount 20 mg and said second amount is 60 mg, (xiv) said first amount 20 mg and said second amount is 80 mg, (xv) said first amount 40 mg and said second amount is 2.5 mg, (xvi) said first amount 40 mg and said second amount is 5 mg, (xvii) said first amount 40 mg and said second amount is 10 mg, (xviii) said first amount 40 mg and said second amount is 20 mg, (xix) said first amount 40 mg and said second amount is 40 mg, (xx) said first amount 40 mg and said second amount is 60 mg, or (xxi) said first amount 40 mg of olmesartan and said second amount is 80 mg.

15. The method as defined in claim 14, wherein said first amount is from about 20-40 mg per day and said second amount is from about 10-20 mg/day.

16. The method of claim 14, wherein the patient is a nonresponder.

17. The method of claim 14, wherein the patient is a partial responder.

18. The method of claim 12, wherein the diuretic is hydrochlorothiazide or chlorthalidone.

19. The method of claim 18, wherein the hydrochlorothiazide is administered in an amount from about 20-50 mg/day.

20. The method of claim 11, wherein the wherein the angiotensin II receptor blocker is irbesartan.

21. The method of claim 20, wherein (i) said first amount is 75 mg and said second amount is 2.5 mg, (ii) said first amount is 75 mg and said second amount is 5 mg, (iii) said first amount is 75 mg and said second amount is 10 mg, (iv) said first amount is 75 mg and said second amount is 20 mg, (v) said first amount is 75 mg and said second amount is 40 mg, (vi) said first amount is 75 mg and said second amount is 60 mg, (vii) said first amount is 75 mg and said second amount is 80 mg, (viii) said first amount is 150 mg and said second amount is 2.5 mg, (ix) said first amount is 150 mg and said second amount is 5 mg, (x) said first amount is 150 mg and said second amount is 10 mg, (xi) said first amount is 150 mg and said second amount is 20 mg, (xii) said first amount is 150 mg and said second amount is 40 mg, (xiii) said first amount is 150 mg and said second amount is 60 mg, (xiv) said first amount is 150 mg and said second amount is 80 mg, (xv) said first amount is 300 mg and said second amount is 2.5 mg, (xvi) said first amount is 300 mg and said second amount is 5 mg, (xvii) said first amount is 300 mg and said second amount is 10 mg, (xviii) said first amount is 300 mg and said second amount is 20 mg, (xix) said first amount is 300 mg and said second amount is 40 mg, (xx) said first amount is 300 mg and said second amount is 60 mg, or (xxi) said first amount is 300 mg and said second amount is 80 mg.

22. The method of claim 21, wherein said first amount is from about 150-300 mg per day and said second amount is from about 10-20 mg per day.

23. The method of claim 21, wherein the patient is a non-responder.

24. The method of claim 21, wherein the patient is a partial responder.

25. A method for treating hypertension in a patient in need thereof, wherein said patient has been previously determined to be responsive to at least one of lercanidipine monotherapy or olmesartan medoxomil monotherapy but has ceased being adequately responsive thereto, said method comprising administering to said patient a first amount of olmesartan medoxomil and a second amount of lercanidipine in combination, wherein said first amount and said second amount are in combination effective to reduce said patient's blood pressure by at least a predetermined increment.

26. The method of claim 25, which method further comprises administrating a diuretic.

27. The method of claim 26, wherein the diuretic is hydrochlorothiazide or chlorthalidone.

28. The method of claim 27, wherein the hydrochlorothiazide is administered in an amount from about 20-50 mg/day.

29. The method as defined in claim 25, wherein (i) said first amount is 10 mg and said second amount is 2.5 mg, (ii) said first amount 10 mg and said second amount is 5 mg, (iii) said first amount is 10 mg and said second amount is 10 mg, (iv) said first amount is 10 mg and said second amount is 20 mg, (v) said first amount 10 mg and said second amount is 40 mg, (vi) said first amount 10 mg and said second amount is 60 mg, (vii) said first amount 10 mg and said second amount is 80 mg, (viii) said first amount 20 mg and said second amount is 2.5 mg, (ix) said first amount 20 mg and said second amount is 5 mg, (x) said first amount 20 mg and said second amount is 10 mg, (xi) said first amount 20 mg and said second amount is 20 mg, (xii) said first amount 20 mg and said second amount is 40 mg, (xiii) said first amount 20 mg and said second amount is 60 mg, (xiv) said first amount 20 mg and said second amount is 80 mg, (xv) said first amount 40 mg and said second amount is 2.5 mg, (xvi) said first amount 40 mg and said second amount is 5 mg, (xvii) said first amount 40 mg and said second amount is 10 mg, (xviii) said first amount 40 mg and said second amount is 20 mg, (xix) said first amount 40 mg and said second amount is 40 mg, (xx) said first amount 40 mg and said second amount is 60 mg, or (xxi) said first amount 40 mg of olmesartan and said second amount is 80 mg.

30. The method as defined in claim 29, wherein said first amount is from about 20-40 mg per day and said second amount is from about 10-20 mg/day.

31. A method for treating hypertension in a patient in need thereof, wherein said patient has been previously determined to be responsive to at least one of lercanidipine monotherapy or irbesartan monotherapy but has ceased being adequately responsive thereto, said method comprising administering to said patient a first amount of irbesartan and a second amount of lercanidipine in combination, wherein said first amount and said second amount are in combination effective to reduce said patient's blood pressure by at least a predetermined increment.

32. The method of claim 31, wherein (i) said first amount is 75 mg and said second amount is 2.5 mg, (ii) said first amount is 75 mg and said second amount is 5 mg, (iii) said first amount is 75 mg and said second amount is 10 mg, (iv) said first amount is 75 mg and said second amount is 20 mg, (v) said first amount is 75 mg and said second amount is 40 mg, (vi) said first amount is 75 mg and said second amount is 60 mg, (vii) said first amount is 75 mg and said second amount is 80 mg, (viii) said first amount is 150 mg and said second amount is 2.5 mg, (ix) said first amount is 150 mg and said second amount is 5 mg, (x) said first amount is 150 mg and said second amount is 10 mg, (xi) said first amount is 150 mg and said second amount is 20 mg, (xii) said first amount is 150 mg and said second amount is 40 mg, (xiii) said first amount is 150 mg and said second amount is 60 mg, (xiv) said first amount is 150 mg and said second amount is 80 mg, (xv) said first amount is 300 mg and said second amount is 2.5 mg, (xvi) said first amount is 300 mg and said second amount is 5 mg, (xvii) said first amount is 300 mg and said second amount is 10 mg, (xviii) said first amount is 300 mg and said second amount is 20 mg, (xix) said first amount is 300 mg and said second amount is 40 mg, (xx) said first amount is 300 mg and said second amount is 60 mg, or (xxi) said first amount is 300 mg and said second amount is 80 mg.

33. The method of claim 32, wherein said first amount is from about 150-300 mg per day and said second amount is from about 10-20 mg per day.

34. A method of treating hypertension in a patient with tachycardia in need thereof, said method comprising administering a first amount of an angiotension II receptor blocker and a second amount of lercanidipine, wherein said amounts in combination are effective to reduce or eliminate the tachycardia, and are effective to reduce or eliminate hypertension in said patient by at least a predetermined increment.

35. The method of claim 34, wherein the tachycardia is a side effect in the patient with hypertension, and wherein patient was administered a first amount of lercanidipine as monotherapy for the treatment of said hypertension prior to said administration of the first amount of lercanidipine and the second amount of an angiotensin II receptor blocker.

36. The method of claim 34, which method further comprises administering a diuretic.

37. The method of claim 36, wherein the diuretic is hydrochlorothiazide or chlorthalidone.

38. The method of claim 37, wherein the hydrochlorothiazide is administered in an amount from about 20-50 mg/day.

39. The method of claim 34, the angiotensin II receptor blocker is olmesartan medoxomil.

40. The method as defined in claim 39, wherein (i) said first amount is 10 mg and said second amount is 2.5 mg, (ii) said first amount 10 mg and said second amount is 5 mg, (iii) said first amount is 10 mg and said second amount is 10 mg, (iv) said first amount is 10 mg and said second amount is 20 mg, (v) said first amount 10 mg and said second amount is 40 mg, (vi) said first amount 10 mg and said second amount is 60 mg, (vii) said first amount 10 mg and said second amount is 80 mg, (viii) said first amount 20 mg and said second amount is 2.5 mg, (ix) said first amount 20 mg and said second amount is 5 mg, (x) said first amount 20 mg and said second amount is 10 mg, (xi) said first amount 20 mg and said second amount is 20 mg, (xii) said first amount 20 mg and said second amount is 40 mg, (xiii) said first amount 20 mg and said second amount is 60 mg, (xiv) said first amount 20 mg and said second amount is 80 mg, (xv) said first amount 40 mg and said second amount is 2.5 mg, (xvi) said first amount 40 mg and said second amount is 5 mg, (xvii) said first amount 40 mg and said second amount is 10 mg, (xviii) said first amount 40 mg and said second amount is 20 mg, (xix) said first amount 40 mg and said second amount is 40 mg, (xx) said first amount 40 mg and said second amount is 60 mg, or (xxi) said first amount 40 mg of olmesartan and said second amount is 80 mg.

41. The method as defined in claim 40, wherein said first amount is from about 10-20 mg/day and said second amount is from is from about 20-40 mg per day.

42. The method of claim 34, the angiotensin II receptor blocker is irbesartan.

43. The method of claim 42, wherein (i) said first amount is 75 mg and said second amount is 2.5 mg, (ii) said first amount is 75 mg and said second amount is 5 mg, (iii) said first amount is 75 mg and said second amount is 10 mg, (iv) said first amount is 75 mg and said second amount is 20 mg, (v) said first amount is 75 mg and said second amount is 40 mg, (vi) said first amount is 75 mg and said second amount is 60 mg, (vii) said first amount is 75 mg and said second amount is 80 mg, (viii) said first amount is 150 mg and said second amount is 2.5 mg, (ix) said first amount is 150 mg and said second amount is 5 mg, (x) said first amount is 150 mg and said second amount is 10 mg, (xi) said first amount is 150 mg and said second amount is 20 mg, (xii) said first amount is 150 mg and said second amount is 40 mg, (xiii) said first amount is 150 mg and said second amount is 60 mg, (xiv) said first amount is 150 mg and said second amount is 80 mg, (xv) said first amount is 300 mg and said second amount is 2.5 mg, (xvi) said first amount is 300 mg and said second amount is 5 mg, (xvii) said first amount is 300 mg and said second amount is 10 mg, (xviii) said first amount is 300 mg and said second amount is 20 mg, (xix) said first amount is 300 mg and said second amount is 40 mg, (xx) said first amount is 300 mg and said second amount is 60 mg, or (xxi) said first amount is 300 mg and said second amount is 80 mg.

44. The method of claim 43, wherein said first amount is from about 10-20 mg per day and said second amount is from about 150-300 mg per day.

45. A pharmaceutical composition for treatment of hypertension comprising a first amount of olmesartan medoxomil and a second amount of lercanidipine said first and second amounts in combination being effective to reduce blood pressure of a patient in need of treatment to within an acceptable value.

46. The composition as defined in claim 45, wherein said first amount is from about 10-40 mg per day.

47. The composition as defined in claim 46, wherein said first amount is from about 20-40 mg per day.

48. The composition as defined in claim 45, wherein said second amount is from about 2.5-80 mg per day.

49. The composition as defined in claim 48, wherein said second amount is from about 10-40 mg per day.

50. The composition as defined in claim 45, wherein (i) said first amount is 10 mg and said second amount is 2.5 mg, (ii) said first amount 10 mg and said second amount is 5 mg, (iii) said first amount is 10 mg and said second amount is 10 mg, (iv) said first amount is 10 mg and said second amount is 20 mg, (v) said first amount 10 mg and said second amount is 40 mg, (vi) said first amount 10 mg and said second amount is 60 mg, (vii) said first amount 10 mg and said second amount is 80 mg, (viii) said first amount 20 mg and said second amount is 2.5 mg, (ix) said first amount 20 mg and said second amount is 5 mg, (x) said first amount 20 mg and said second amount is 10 mg, (xi) said first amount 20 mg and said second amount is 20 mg, (xii) said first amount 20 mg and said second amount is 40 mg, (xiii) said first amount 20 mg and said second amount is 60 mg, (xiv) said first amount 20 mg and said second amount is 80 mg, (xv) said first amount 40 mg and said second amount is 2.5 mg, (xvi) said first amount 40 mg and said second amount is 5 mg, (xvii) said first amount 40 mg and said second amount is 10 mg, (xviii) said first amount 40 mg and said second amount is 20 mg, (xix) said first amount 40 mg and said second amount is 40 mg, (xx) said first amount 40 mg and said second amount is 60 mg, or (xxi) said first amount 40 mg of olmesartan and said second amount is 80 mg.

51. A pharmaceutical composition for treatment of hypertension comprising a first amount of irbesartan and a second amount of lercanidipine said first and second amounts in combination being effective to reduce blood pressure of a patient in need of treatment to within an acceptable value.

52. The composition as defined in claim 51, wherein said first amount is from about 50-300 mg per day.

53. The composition as defined in claim 52, wherein said first amount is from about 150-300 mg per day.

54. The composition as defined in any one of claims 51, wherein said second amount is from about 2.5-80 mg per day.

55. The method as defined in claim 54, wherein said second amount is from about 10-40 mg per day.

56. The composition as defined in claim 51, wherein (i) said first amount is 75 mg and said second amount is 2.5 mg, (ii) said first amount is 75 mg and said second amount is 5 mg, (iii) said first amount is 75 mg and said second amount is 10 mg, (iv) said first amount is 75 mg and said second amount is 20 mg, (v) said first amount is 75 mg and said second amount is 40 mg, (vi) said first amount is 75 mg and said second amount is 60 mg, (vii) said first amount is 75 mg and said second amount is 80 mg, (viii) said first amount is 150 mg and said second amount is 2.5 mg, (ix) said first amount is 150 mg and said second amount is 5 mg, (x) said first amount is 150 mg and said second amount is 10 mg, (xi) said first amount is 150 mg and said second amount is 20 mg, (xii) said first amount is 150 mg and said second amount is 40 mg, (xiii) said first amount is 150 mg and said second amount is 60 mg, (xiv) said first amount is 150 mg and said second amount is 80 mg, (xv) said first amount is 300 mg and said second amount is 2.5 mg, (xvi) said first amount is 300 mg and said second amount is 5 mg, (xvii) said first amount is 300 mg and said second amount is 10 mg, (xviii) said first amount is 300 mg and said second amount is 20 mg, (xix) said first amount is 300 mg and said second amount is 40 mg, (xx) said first amount is 300 mg and said second amount is 60 mg, or (xxi) said first amount is 300 mg and said second amount is 80 mg.

57. The method of claim 4, wherein said second amount of 80 mg is administered in a modified release formulation.

58. The method of claim 9, wherein said second amount of 80 mg is administered in a modified release formulation.

59. The method of claim 14, wherein said second amount of 80 mg is administered in a modified release formulation.

60. The method of claim 21, wherein said second amount of 80 mg is administered in a modified release formulation.

61. The method of claim 29, wherein said second amount of 80 mg is administered in a modified release formulation.

62. The method of claim 32, wherein said second amount of 80 mg is administered in a modified release formulation.

63. The method of claim 40, wherein said second amount of 80 mg is administered in a modified release formulation.

64. The method of claim 43, wherein said second amount of 80 mg is administered in a modified release formulation.

65. The method of claim 50, wherein said second amount of 80 mg is administered in a modified release formulation.

66. The method of claim 56, wherein said second amount of 80 mg is administered in a modified release formulation.

67. A method for treating hypertension in a patient in need thereof, said method comprising administering to said patient a first amount of an angiotensin II receptor blocker, a second amount of lercanidipine, and a third amount of a diuretic, wherein said amounts in combination are effective to reduce blood pressure in said patient by at least a predetermined increment; wherein each of said first amount, said second amount, and said third amount, administered individually is ineffective to produce a reduction in blood pressure in said patient or said reduction in blood pressure is less than said predetermined increment.

68. The method of claim 67, wherein the diuretic is hydrochlorothiazide.

69. The method of claim 68, wherein the hydrochlorothiazide is administered in an amount from about 20-50 mg/day.

70. The method of claim 67, wherein the diuretic is chlorthalidone.

71. The method of claim 70, wherein the chlorthalidone is administered in an amount from about 25-50 mg/day.
